(19) United States
(12) Patent Application Publication
(10) Pub. No.: US 2025/0103461 A1
(43) Pub. Date: Mar. 27, 2025

(54) METHOD OF VERIFYING A PROGRAM CODE OF A SYNTHESIS COMPUTER PROGRAM

(71) Applicant: Hoffmann-La Roche Inc., Little Falls, NJ (US)

(72) Inventors: Marek Stanislaw Komisarski, Basel (CH); Pascal Mueller, Basel (CH); Martin Olbrich, Basel (CH); Maxim Popov, Basel (CH); Johanna Romano, Basel (CH); Andreas Schuster, Basel (CH)

(21) Appl. No.: 18/974,519

(22) Filed: Dec. 9, 2024

Related U.S. Application Data
(63) Continuation of application No. PCT/EP2023/065183, filed on Jun. 7, 2023.

(30) Foreign Application Priority Data
Jun. 8, 2022 (EP) 22177729.5

(51) Int. Cl. G06F 11/3604; G06F 40/205

(52) U.S. Cl. CPC G06F 11/3604; G06F 40/205

(57) ABSTRACT

A method of verifying a program code of a synthesis computer program is disclosed. The synthesis computer program is configured for computer-controlling an automatic synthesizer (114) to automatically synthesize at least one oligonucleotide, the synthesis computer program having a plurality of program cycles for computer-controlling the automatic synthesizer (114) to sequentially synthesize the oligonucleotide by using at least one sequence of synthesis cycles. The method comprises:
i. applying an automatic parsing procedure to the program code of the synthesis computer program, the automatic parsing procedure comprising automatically searching for parameter values of parameters of at least one predetermined list of parameters of interest in the program cycles of the synthesis computer program; and
ii. automatically assembling a matrix of parameters comprising, for the program cycles of the synthesis computer program, the parameters of interest and the corresponding parameter values of the parameters of interest.

Further disclosed is a computer program and a computer-readable storage medium for performing the method of verifying a program code of a synthesis computer program, a system (110) for verifying a program code of a synthesis computer program and a method of synthesizing at least one oligonucleotide.

### TECHNICAL FIELD

The present invention refers to a method and a system for verifying a program code of a synthesis computer program. The invention further refers to a computer program and a computer-readable storage medium for performing the method. Further, the invention refers to a method of synthesizing at least one oligonucleotide. The methods, the system, the computer program and the computer-readable storage medium may be used for research or manufacturing, specifically industrial scale manufacturing, of oligonucleotides, specifically by means of solid phase synthesis.

### BACKGROUND ART

The principles of the oligonucleotide synthesis are well known in the art (see e.g. Oligonucleotide synthesis; Wikipedia, the free encyclopedia; https://en.wikipedia.org/wiki/Oligonucleotide synthesis, of May 30, 2022). Synthesizing oligonucleotides, for example by means of solid phase synthesis, generally requires sequentially performing multiple synthesis cycles. The synthesis can be carried out automatically using computer-controlled synthesizers configured for automatically performing the sequence of synthesis cycles. The synthesizer may specifically be configured for performing the sequence of synthesis cycles according to a computer program controlling a functionality of the synthesizer. The computer program may comprise commands for controlling the synthesizer according to a given synthesis plan indicating specific synthesis parameters, related to reagents, solvents, flow rates, volumes, time intervals and others, to be set at the synthesizer in different synthesis cycles.

In general, when using computer programs for controlling synthesis of oligonucleotides, deviations between the computer program and the synthesis plan are difficult to detect since the computer program often comprises complex sequences of commands, specifically having a plurality of functions and sub-routines. Deviations between the computer program and the synthesis plan, for example deviations regarding reagents or solvents to be used in a specific synthesis cycle and/or deviations in amount, flow and/or delivery time of reagents or solvents used in specific synthesis cycle, may disable the synthesis of the oligonucleotide or may lead to the production of incorrect and/or sub-standard quality oligonucleotides.

Since oligonucleotides specifically may be used for pharmaceutical purposes, it is desirable to carefully verify any computer program for controlling the synthesis, before the synthesis is actually started. As outlined above, however, the complexity of the many computer programs which typically are used for controlling syntheses renders the verification rather difficult. Specifically, the use of functions and routines within the program aggravates checking the completeness as well as checking the values of the many parameters to be used in the process of synthesis.

### Problem to be Solved

It is therefore desirable to provide methods and devices that at least partially address above-mentioned technical challenges. Specifically, methods and devices shall be proposed which allow for a verification of computer programs used for oligonucleotide synthesis and which provide for a reliable and robust oligonucleotide synthesis.

### SUMMARY

This problem is addressed by a method of verifying a program code of a synthesis computer program, a system for verifying a program code of a synthesis computer program, a computer program and a computer-readable storage medium for performing said method and by a method of synthesizing at least one oligonucleotide with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a method of verifying a program code of a synthesis computer program is disclosed.

The term “verifying” as used herein is a broad term and is to be given its ordinary and customary meaning to a person of ordinary skill in the art and is not to be limited to a special or customized meaning. The term specifically may refer, without limitation, to a process of assessing one or more specifications of a program code. Specifically, the process of verifying the program code, also known as “verification” or “software verification”, may comprise assessing an implementation of one or more specifications of the program code. The specification of the program code may comprise one or more of: an outcome, specifically a predefined outcome or result of the program code after being executed, required computing resources for executing the program code, time needed for executing the program code, parameters and parameter values used during execution of the program code or used in the program code, and the like. In other words, the process of verifying may comprise determining whether one or more of the specifications of the program code satisfies or meets one or more defined conditions, e.g. conditions defined prior to program code creation and/or conditions which are implemented or defined when the program is executed, e.g. by calling the program code with a desired set of parameters and/or parameter values. A result of the process of verifying the program code may comprise one or more findings that the specifications of the program code are fully implemented, partially implemented and/or not implemented. As an example, the program code of the synthesis computer program may be created according to a given synthesis plan. The process of verifying may comprise determining if the program code is suitable for implementing the given synthesis plan on the synthesizer, e.g. by performing a given sequence of synthesis cycles, using correct reagents, using a correct sequence of reagents, using a correct sequence of solvents, using correct starting materials, using correct solvents and/or flow rates, etc., In particular, the process of verifying the program code may comprise determining if a correct sequence of occurrence, e.g. a correct sequence of occurrence of reactants and/or solvents at specific synthesis cycles, is used according to a given synthesis plan.

The term “program code” as used herein is a broad term and is to be given its ordinary and customary meaning to a person of ordinary skill in the art and is not to be limited to a special or customized meaning. The term specifically may refer, without limitation, to a sequence of instructions that can be executed or interpreted by a computer. The program code, when being executed by a computer, specifically by a central processing unit (CPU) of the computer, more specifically a central programming unit of the computer, may cause the computer to execute the instructions comprised by the program code. The program code may be written in a programming or scripting language, such as C, C #, C++, Java, python, R, Fortran, JavaScript, HTML or the like. The program code may specifically be in a human-readable form and, thus, may also be referred to as “source code”. For executing the program code on the computer, the program code may be transferred by a programming language-specific compiler into machine-readable instructions. The instructions of the program code may comprise instructions which cause the computer to perform a specific function and/or declarations, e.g. for defining functions, variables, constants and/or classes comprised by the program code. Alternatively or additionally, the program code may comprise supplementary information, such as headers placed in front of the sequence of instructions comprised by the program code, comments or further information in between the instructions of the program code or the like. Alternatively or additionally, the program code may comprise an incomplete program code, such as a program code having gaps to be filled with specific parameter values, wherein the program code may be completed by performing the method.

The sequence of instructions comprised by the program code may specifically be comprised by a text section following the header. The program code may comprise a single sequence of instructions, which, when the program code is executed by the computer, are performed in a given order, e.g. starting with a first instruction and ending with a last instruction. Alternatively or additionally, the program code may comprise multiple blocks of program code, wherein each block of program code comprises a sequence of instructions. The sequence of instructions within each block of program code may be executed in the given order. However, the program code may comprise within a main sequence of instructions references to one or more other blocks of program code. Thus, when executing the main sequence of instructions of the program code, the computer may jump to the referred block of program code and may execute the referred block of program code. Further, the program code may comprise multiple references to the same block of program code.

The program code specifically may be a method file, specifically a Unicorn method file. Additionally or alternatively, the program code may be a binary program code.

The term “computer program” as used herein is a broad term and is to be given its ordinary and customary meaning to a person of ordinary skill in the art and is not to be limited to a special or customized meaning. The term specifically may refer, without limitation, to a functional entity comprising one or more software applications for enabling a computer to carry out at least one functionality. Specifically, the computer program may denote an algorithm which is performed by the computer, when the computer executes the program code of the computer program. In other words, the program code may comprise the computer readable instructions, whereas the computer program may constitute the manifestation of what is actually done by the computer when the computer executes the computer readable instructions.

The term “computer” as used herein is a broad term and is to be given its ordinary and customary meaning to a person of ordinary skill in the art and is not to be limited to a special or customized meaning. The term specifically may refer, without limitation, to a device or system configured for electronic data processing. Specifically, the computer, which may comprise a single computer, a distributed computer or a computer network, may also comprise one or more processors for data processing. The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processor specifically may be configured, such as by software programming, for performing one or more evaluation operations.

The term “synthesis computer program”, as used herein, is a broad term and is to be given its ordinary and customary meaning to a person of ordinary skill in the art and is not to be limited to a special or customized meaning. The term specifically may refer, without limitation, to a computer program for enabling a computer to carry out the function of controlling an automatic synthesizer to synthesize at least one oligonucleotide. The program code may be part of or may be comprised by the synthesis computer program. The synthesis computer program may comprise one or more software applications for executing the program code on the computer. For example, the synthesis computer program may comprise at least one compiler for translating the program code into machine-readable instructions. Thus, the synthesis computer program may comprise at least one software application, which when executed by a computer, cause the computer to carry out the instructions given by the program code, e.g. for executing the synthesis of the at least one oligonucleotide on the automatic synthesizer. The synthesis computer program may comprise at least one software application for interacting with one or more of the computer's hardware and the automatic synthesizer. Further, the synthesis computer program may comprise at least one software application for providing a user interface. Via the user interface, the user may be able to input and/or to create the program code for the synthesis of the at least one oligonucleotide on the automatic synthesizer.

The synthesis computer program is configured for computer-controlling an automatic synthesizer to automatically synthesize at least one oligonucleotide. The synthesis computer program has a plurality of program cycles for computer-controlling the automatic synthesizer to sequentially synthesize the oligonucleotide by using at least one sequence of synthesis cycles.

The term “computer-controlling” as used herein is a broad term and is to be given its ordinary and customary meaning to a person of ordinary skill in the art and is not to be limited to a special or customized meaning. The term specifically may refer, without limitation, to a process of controlling one or more devices by means of at least one computer. The controlling may specifically comprise at least one of: setting one or more parameters and/or parameter values at the device to be controlled; monitoring the parameters; determining deviations between set points of the parameters and actual values of the parameters; adapting the set points of the parameters according to the determined deviations. The parameters may be continuous parameters and/or discrete parameters, specifically binary parameters. For example, controlling the automatic synthesizer may comprise turning on and/or off one or more valves of the automatic synthesizer and/or setting the valves to a specific position. Alternatively or additionally, controlling the automatic synthesizer may comprise initiating one or more pumps, specifically setting a flow rate and/or a pump pressure at one or more pumps. The parameters may be set by means of at least one actuator specifically by using electric actuators. The actuator may specifically be configured for moving and/or controlling a mechanism of the automatic synthesizer, for example for moving and/or controlling a valve and/or a pump of the automatic synthesizer, specifically by converting an electronic control signal into mechanical motion. The parameters may be monitored by means of at least one sensor device, for example flow sensors, fluid velocity sensors and/or pressure sensors.

The term “synthesize” as used herein is a broad term and is to be given its ordinary and customary meaning to a person of ordinary skill in the art and is not to be limited to a special or customized meaning. The term specifically may refer, without limitation, to generating at least one covalent connection between two nucleoside monomers of an oligonucleotide by chemical means. Methods for synthesizing oligonucleotides have been known in the art for a long time, the most widely used method being solid-phase phosphoramidite synthesis using phosphoramidite building blocks, in an embodiment derived from protected 2′-deoxynucleosides (dA, dC, dG, and dT), ribonucleosides (A, C, G, and U), or chemically modified nucleosides, e.g. locked nucleic acid (LNA) monomers; exemplary reference may be made e.g. to the Wikipedia citation mentioned above. In an embodiment, synthesis is semi- or fully automated synthesis, in which the synthesis steps may be controlled partly or completely by an automatic synthesizer. In an embodiment, synthesis is automated synthesis as specified herein below.

The term “synthesizer” as used herein is a broad term and is to be given its ordinary and customary meaning to a person of ordinary skill in the art and is not to be limited to a special or customized meaning. The term specifically may refer, without limitation, to any device adapted to perform a synthesis as specified herein, for example as will be outlined in further detail below, in an embodiment in an automated manner. The synthesizer may typically be an automatic synthesizer which is capable to synthesize oligonucleotides on the desired scale. Automatic synthesizers are e.g. the oligoplot synthesizers commercialized by the company Cytiva®. The synthesizer may comprise at least one synthesis column having at least one stationary phase material, such as at least one solid phase material. The synthesizer may further comprise at least one pump, connectors to a plurality of chemical supplies and/or a system of switchable valves for selectively supplying chemicals, specifically solvent and/or reactants, to the column. In particular, the synthesizer may comprise an amidite pump; an amidite valve; a solvent/reagent pump; a solvent/reagent valve; a system of switchable valves for feeding liquid from the column into predetermined receptacles; at least one tempering element for setting the column to at least one predetermined temperature; a column inlet valve for admitting liquid flow into the column; a column outlet valve for admitting liquid flow from the column; a recycling bypass, specifically for recycling liquid over the column, such as by connecting a column outlet valve to a column inlet valve by means of at least one valve; and/or a waste valve for directing liquid from the column into at least one waste system.

The automatic synthesizer may further comprise at least one processor as specified herein above, wherein said processor is adapted to execute the synthesis computer program. Thus, the processor in an embodiment is operably linked to at least a valve and/or a pump of the automatic synthesizer. The skilled person knows how to operably connect the microprocessor to the other units of the automatic synthesizer; appropriate automatic synthesizers are known in the art and are commercially available.

The terms “automatic” or “automatically”, as used herein, are broad terms and are to be given their ordinary and customary meaning to a person of ordinary skill in the art and are not to be limited to a special or customized meaning. The terms specifically may refer, without limitation, to a property of a device being at least partially controlled by means of at least one computer, specifically comprising one or more processors, and/or of a process being at least partially performed by means of at least one computer and/or machine, in particular without manual action and/or interaction with a user. Thus, the term “automatic” may refer to a situation in which the automatic device, for example the automatic synthesizer, may be configured for performing at least one task fully or at least partially without the need of human interaction, and/or may refer to a situation in which the automatic device is at least partially controlled by means of at least one computer such that parts of the functions of the automatic device are controlled by the computer. As an example, the automatic synthesis of the at least one oligonucleotide may comprise, once the synthesizer is prepared, the devices and the materials for the synthesis are provided and the automatic synthesis is started, a full or at least partial synthesis of the desired oligonucleotide or at least a predecessor or intermediate product thereof, preferably without the need of human interaction. Alternatively, this term may also refer to a situation in which the automatic device, for example the automatic synthesizer, may be configured for being completely controlled by means of at least one computer such that all of the functions of the automatic device are controlled by the computer. Similarly, the term “automatic” as used in context of a process, for example automatically synthesizing the at least one oligonucleotide, may refer to a situation in which the process is performed or controlled fully or partially by means of at least one computer such that a part of the process steps are performed by the computer, whereas other process steps are performed by manual interaction and/or by a user. Alternatively or additionally, automatically performing a process may comprise completely performed by at least one computer, preferably without manual action and/or interaction with a user.

In an embodiment, oligonucleotide synthesis is a solid-phase synthesis, wherein the oligonucleotide being assembled is covalently bound, via its 3′-terminal hydroxy group, to a solid support material and remains attached to it over the entire course of the chain assembly. Suitable supports are commercially available, e.g. macroporous polystyrene supports like the Primer support 5G from GE Healthcare, the NittoPhase®HL support from Kinovate, or controlled glass supports.

The oligonucleotide synthesis in an embodiment is a stepwise addition of nucleotide residues to the 5′-terminus of the growing chain until the desired sequence is assembled. In an embodiment, each addition is referred to as a synthetic cycle and may in particular comprise the following steps:
a1) de-blocking the protected hydroxyl group on the solid support or on the latest nucleoside attached to the growing chain,
a2) coupling the first nucleoside as activated phosphoramidite with the free hydroxyl group on the solid support
a3) oxidizing or sulfurizing the respective P-linked nucleoside to form the respective phosphodiester (P═O) or the respective phosphorothioate (P═S);
a4) optionally, capping any unreacted hydroxyl groups on the solid support;
a5) de-blocking the 5′ hydroxyl group of the latest nucleoside attached to the growing chain;
a6) coupling the next nucleoside as activated phosphoramidite to the growing chain;
a7) oxidizing or sulfurizing the respective P-linked dinucleoside to form the respective phosphodiester (P═O) or the respective phosphorothioate (P═S);
a8) optionally, capping any unreacted 5′ hydroxyl groups;
a9) repeating the previous steps a5 to a8 until the desired sequence is assembled.

The concluding steps after the chain has been completed are known to the skilled person, and include in an embodiment backbone deprotection and/or cleavage from the resin, wherein cleavage from therein can be e.g. performed with concentrated aqueous ammonia. The protecting groups on the phosphate and the nucleotide base are also removed within this cleavage procedure.

In accordance with the above, the term “synthesis cycle” relates to any sequence of steps performed in order to add one nucleotide moiety to the oligonucleotide chain. The synthesis cycle may be an initiation cycle, i.e. a sequence of steps causing the first nucleotide moiety to become covalently attached to the solid phase of the synthesizer. Such an initiation cycle may e.g. comprise the steps described as steps a1) to a4) herein above. The synthesis cycle may also be an elongation cycle, i.e. a sequence of steps causing elongation of the oligonucleotide chain and, optionally, preparation of the oligonucleotide for the next elongation cycle. An exemplary sequence of steps of an elongation cycle is provided herein as steps a5) to a8).

The term “oligonucleotide” as used herein is a broad term and is to be given its ordinary and customary meaning to a person of ordinary skill in the art and is not to be limited to a special or customized meaning. The term specifically may refer, without limitation, to any chemical molecule comprising at least two nucleoside moieties which are covalently connected. In an embodiment, the oligonucleotide comprises of from 2 to 100 nucleotide moieties, i.e. “has a length” of from 2 to 100 bases, in an embodiment comprises of from 5 to 50 nucleotide moieties, in a further embodiment comprises of from 10 to 40 nucleotide moieties. In an embodiment, oligonucleotides may consist of optionally modified DNA, RNA or LNA nucleotide monomers or combinations thereof. The LNA nucleotide monomers are modified nucleotides which comprise a linker group (referred to as a biradicle or a bridge) between C2′ and C4′ of the ribose sugar ring of a nucleotide. These nucleotides are also termed bridged nucleic acid or bicyclic nucleic acid (BNA) in the literature. Optionally modified as used herein refers to nucleotides modified as compared to the equivalent DNA, RNA or LNA nucleotide by the introduction of one or more modifications of the sugar moiety or the nucleobase moiety. Modifications and appropriate building blocks are known in the art and include, without limitation, e.g. Methoxyethyl (MOE) modifications, fluoro modifications, methoxy modifications, and the like.

As the skilled person will understand, the terms “nucleoside” and “nucleotide” are used herein in a broad sense, since the nucleosides may comprise bases different from the standard bases of the genetic code; also, the sugar moiety of the nucleoside may be a non-standard sugar, like e.g. a 2′ and/or 3′ derivatized ribose moiety, a brigded ribose moiety, a non-ribose moiety, and the like. In an embodiment the modified nucleoside comprises a modified sugar moiety, and may for example comprise one or more 2′ substituted nucleosides and/or one or more LNA nucleosides. Thus, the term modified nucleoside may also be used herein interchangeably with the term “nucleoside analogue” or modified “units” or modified “monomers”. In an embodiment the sugar residue is ribose or 2′-deoxyribose. Nucleosides are as a general rule linked in the oligonucleotide by a phosphodiester (P═O) and/or a phosphorothioate (P═S) internucleoside linkage, which covalently couples two nucleosides together. Accordingly, in some oligonucleotides all internucleoside linkages may consist of a phosphodiester (P═O), in other oligonucleotides all internucleoside linkages may consist of a phosphorothioate (P═S), or in still other oligonucleotides the sequence of internucleoside linkages may vary and comprise both phosphodiester (P═O) and phosphorothioate (P═S) internucleoside. As will be understood from the description herein above, the term “nucleotide” is used herein in a broad sense to relate to a monomer of an oligonucleotide; thus, the nucleotide may e.g. a nucleoside with a 5′ phosphate group (i.e. a nucleotide sensu stricto), but may also be e.g. a phosphorothioate nucleotide.

Moreover, non-nucleoside moieties may also be incorporated into an oligonucleotide, such as dye moieties, including quencher moieties, affinity moieties such as biotin, and/or chelator moieties. Also, the oligonucleotide can be 5′ amino modified which signifies that an amino linker is attached to the 5′ terminal group of the oligonucleotide.

The nucleobase moieties may be indicated by the letter code for each corresponding nucleobase, e.g. A, T, G, C or U, wherein each letter may optionally include modified nucleobases of equivalent function. Modified nucleobases include but are not limited to nucleobases carrying protecting groups such as tert-butylphenoxyacetyl, phenoxyacetyl, benzoyl, acetyl, isobutyryl or dimethylformamidino.

As outlined above, the synthesis computer program has a plurality of program cycles for computer-controlling the automatic synthesizer to sequentially synthesize the oligonucleotide by using the at least one sequence of synthesis cycles. The term “program cycles” as used herein is a broad term and is to be given its ordinary and customary meaning to a person of ordinary skill in the art and is not to be limited to a special or customized meaning. The term specifically may refer, without limitation, to one or more blocks of program code for performing one synthesis cycle and/or one sub step of one synthesis cycle from the sequence of synthesis cycles, as will be outlined in further detail below. The program cycles specifically may be performed repeatedly, with identical or varying parameters, in particular as specified herein above for the synthesis of oligonucleotides. The program cycles may be labeled uniquely in the program code, e.g. by defining a name for the respective program cycle. The program cycles may be defined once in the program code, such as in terms of parameters to be set and/or associated parameter values. The program cycles may be performed by referencing the respective name of the program cycle in the program code. Thus, a specific program cycle may comprise identical parameters, specifically parameter values, for the entire synthesis of the oligonucleotide, even if performed multiple times in the synthesis. The parameters, however, may be assigned differing parameter values in different program cycles, such that, as an example, even though the parameters of the program cycles may fully or partially be identical, the parameter values of the parameters may be different in the differing runs of the program cycles.

The term “sequence of synthesis cycles” as used herein is a broad term and is to be given its ordinary and customary meaning to a person of ordinary skill in the art and is not to be limited to a special or customized meaning. The term specifically may refer, without limitation, to any timely arrangement of sequence cycles as specified herein above. The sequence of synthesis cycles, in an embodiment, comprises at least one initiation cycle, followed by at least one elongation cycle, in an embodiment as specified herein above. As the skilled person will understand, the number of elongation cycles typically corresponds to the number of nucleotide moieties in the desired oligonucleotide minus one.

The term “sequentially” as used herein is a broad term and is to be given its ordinary and customary meaning to a person of ordinary skill in the art and is not to be limited to a special or customized meaning. The term specifically may refer, without limitation, to an ordered performance of multiple process steps. Sequentially performing multiple process steps, for example sequentially synthesizing the oligonucleotide by using the sequence of synthesis cycles, may comprise performing the process steps, specifically the synthesis cycles, one after another in a given order. For example, if a process comprises process steps A, B and C to be performed in the given order, sequentially performing this process may comprise first performing process step A, followed by process step B and followed by process step C. The process steps may not timely overlap with each other. The following process step may be initiated once the previous process step is finished.

The method comprises at least the following steps which, as an example, may be performed in the given order. It shall be noted, however, that a different order is also possible. Further, it is also possible to perform one, more than one or even all of the method steps once or repeatedly. Further, it is possible to perform two or more of the method steps simultaneously or in a timely overlapping fashion. The method may comprise further method steps which are not listed.

The method comprises:
i. applying an automatic parsing procedure to the program code of the synthesis computer program, the automatic parsing procedure comprising automatically searching for parameter values of parameters of at least one predetermined list of parameters of interest in the program cycles of the synthesis computer program; and
ii. automatically assembling a matrix of parameters comprising, for the program cycles of the synthesis computer program, the parameters of interest and the corresponding parameter values of the parameters of interest.

The term “parsing procedure” as used herein is a broad term and is to be given its ordinary and customary meaning to a person of ordinary skill in the art and is not to be limited to a special or customized meaning. The term specifically may refer, without limitation, to a process of analyzing a program code in order to identify one or more contents of the program code and/or in order to analyze the program code into its constituents. The parsing procedure may comprise analyzing the program code for at least one of specific instructions comprised by the program code, specific parameters comprised by the program code and specific parameter values comprised by the program code. The parsing procedure may comprise splitting the program code into single instructions. Specifically, the parsing procedure may take the program code as input and may output parsed instructions comprised in the program code. The parsing procedure may comprise pattern matching, e.g. pattern matching for finding predefined instructions in the program code. The parsing procedure may further comprise extracting items of information from the program code associated with the parsed instructions, for example as parameters and/or parameter values to be set at the automatic synthesizer. As an example, many programming languages such as Python comprise a plurality of parsing commands identifying specific constituents of data, such as a string, e.g. a text string. When the program code is considered a text string, as an example, the parsing procedure may comprise, by using the parsing commands, identifying the content of specific positions of the program code, such as identifying specific parameters and/or specific parameter values of the program code. As an example, even though the program cycles of the computer program may vary, specific parameters and/or specific parameter values may be identifiable by searching for specific keywords within the program code, as will be outlined in further detail below. Additionally or alternatively, other parsing strategies may be used.

As outlined above, the program code may comprise a header and a text section following the header. The method may comprise an automatic detection of the text section in the program code. The automatic parsing procedure of step i. may specifically be performed on the text section.

The parsing procedure may be performed on a complete program code of a synthesis program, e.g. a program file, or on one or more parts thereof. Also, the parsing procedure may only parse a portion of the program cycles of the synthesis computer program, e.g. program cycles comprising a pre-determined keyword or parameter value.

The term “parameter” as used herein is a broad term and is to be given its ordinary and customary meaning to a person of ordinary skill in the art and is not to be limited to a special or customized meaning. The term specifically may refer, without limitation, to a variable and/or adjustable setting of a process and/or of a device. Specifically, the parameter may be variable and/or adjustable by controlling means, for example by means of computer control. The parameter may be a continuous parameter, such as flow rate, pressure, level, temperature, time or the like, or a discrete parameter, such as a state of a valve, a pump or a flow restrictor. The program code comprising instructions for setting parameters at the automatic synthesizer may comprise a plurality of parameters, specifically a plurality of different parameters. The parameters in the program code may be associated with a unit, specifically a unit being explicitly defined in a block of program code or a unit being defined by referencing to another explicit definition of the unit in another block of program code.

The term “parameter value” as used herein is a broad term and is to be given its ordinary and customary meaning to a person of ordinary skill in the art and is not to be limited to a special or customized meaning. The term specifically may refer, without limitation, to a numerical and/or alphanumerical indication of a parameter. Specifically, the parameter value may be a numerical and/or alphanumerical indication of the parameter comprised in the program code. For example, the parameter may comprise a flow rate, a pressure, a level, a temperature or the like. The parameter value may be a numerical indication of the flow rate, pressure, level or temperature being set at the automatic synthesizer. As another example, the parameter may comprise a state of a valve, pump or flow restrictor. The parameter value may be a numerical indication of the state of the valve, pump or flow restrictor, such as “0” or “1” and/or a position of the valve. Alternatively, the parameter value may be an alphanumerical indication of the state of the valve, pump or flow restrictor, such as “on” or “off”. The parameter value may also indicate a specific reagent or solvent used at the automatic synthesizer. Thus, the parameter value may comprise an indication of the reagent or solvent, such as compound “A”, “B”, “C” or the like.

The term “parameter of interest” as used herein is a broad term and is to be given its ordinary and customary meaning to a person of ordinary skill in the art and is not to be limited to a special or customized meaning. The term specifically may refer, without limitation, to a parameter being searched for. One or more parameters of interest may be predefined, e.g. by the method of verifying the program code itself and/or by a user initiating the method of verifying the program code. Specifically, the parameters of interest may comprise one or more parameters, specifically predefined parameters, being searched for in the automatic parsing procedure. The parameters of interest may specifically have influence on the synthesis of the at least one oligonucleotide. Thus, the parameters of interest may comprise specific settings at the automatic synthesizer which influence the synthesis of the oligonucleotide. The parameters of interest may be defined and/or known prior to the automatic parsing procedure, specifically such that the parameters of interest can be collected in the predetermined list.

The term “predetermined list” as used herein is a broad term and is to be given its ordinary and customary meaning to a person of ordinary skill in the art and is not to be limited to a special or customized meaning. The term specifically may refer, without limitation, to a collection of predefined items. The pre-definition may be performed before starting the method of verifying, or, alternatively, may be performed during the method of verifying itself, e.g. by an interaction with a user, e.g. a computer request to the user to enter or select the components of the list. Specifically, the predetermined list of parameters of interest may comprise a collection of predefined parameters of interest. In particular, however, even though the pre-definition may be performed also after starting the method, the predetermined list of parameters of interest specifically may be defined prior to performance of the automatic parsing procedure.

The predetermined list of parameters of interest specifically may comprise at least one parameter of interest selected from the group consisting of: a type of a chemical, specifically a reactant and/or a solvent; a volume of a chemical, specifically a reactant and/or a solvent; a flow rate of a chemical, specifically a reactant and/or a solvent; a temperature of a chemical, specifically a reactant and/or a solvent; a pump pressure of at least one pump; a length of the synthesis computer program, specifically a length of a synthesis cycle, such as a length of a synthesis cycle in minutes and/or milliliters; a duration of the synthesis computer program, specifically a duration of a synthesis cycle.

Further, in the automatic parsing procedure, a list of keywords may be assembled corresponding to the list of parameters of interest. The keywords of the list of keywords may be known to be used in conjunction with the corresponding parameters of interest of the list of parameters of interest in the program code of the synthesis computer program. The parameter values of the parameters of interest may be known to be located in the program code in a predetermined relative location relative to the keywords. The automatic parsing procedure may further comprise reading the corresponding parameter values of the parameters of interest from the predetermined relative location of the corresponding keywords. As outlined above, many computer languages such as Python provide for commands which allow for searching data, such as text strings, for specific keywords and for reading information which is located in a predefined position relative to the identified keywords, such as parameter values which are known to be in a specific position relative to the keyword, e.g. at a predefined offset from the keywords.

For example, the list of keywords may comprise one or more of the following keywords:
keyword “block”, e.g. for reading calls to a specific block of program code or subroutine;
keyword “base”, e.g. for reading units of parameters set in the current block of program code, such as volume, pressure or the like;
keyword “amidite”, e.g. for reading amidites set in the current block of program code;
keyword “flow_AB”, e.g. for reading flow rate values at pumps A and B, for example “Flow_AB 0.1 {ml/min}, 0.8 {ml/min}” setting the flow rate at pump A to 0.1 ml/min and flow rate at pump B to 0.8 ml/min;
keywords “solvent_A” and/or “solvent_B”, e.g. for reading solvents for pump A and/or B; and
keyword “V”, e.g. for reading valve settings, for example “V5 2” setting valve no. 5 to position no. 2.

However, further keywords may be possible.

The term “assembling” as used herein is a broad term and is to be given its ordinary and customary meaning to a person of ordinary skill in the art and is not to be limited to a special or customized meaning. The term specifically may refer, without limitation, to a process of combining a plurality of items of information, specifically from different locations, into a single item of information and/or into a single location. Specifically, the process of assembling may comprise one or more intermediate process steps. For example, the assembling may comprise collecting the plurality of items of information from one or more information sources, specifically from the program code. Thus, the assembling may comprise the search for the items of information in a complex entity, such as, in the present case, the program code, and collecting these items of information, such as by assigning these items of information to a specific region of data storage, visualizing these items of information in a specific way, assigning these items of information to one or more dedicated variables or the like. The assembling may further comprise re-arranging the collected items of information into a given and/or predefined order. The assembling may comprise may further comprise providing the combined single item of information, such as by displaying the combined single item of information to a user. In particular, the assembling may comprise combining the parameters of interest and the corresponding parameter values into a single item of information, in particular into the matrix of parameters.

The term “matrix of parameters” as used herein is a broad term and is to be given its ordinary and customary meaning to a person of ordinary skill in the art and is not to be limited to a special or customized meaning. The term specifically may refer, without limitation, to an arbitrary one dimensional, two-dimensional or multidimensional array or table of parameters or parameter values. Specifically, the matrix of parameters may be a rectangular and/or two-dimensional array or table of parameters arranged in rows and columns. Therein, as an example, rows may denote the program cycles and columns may denote specific parameters of the parameters of interest or vice versa. Other notations are also feasible.

Also in an embodiment, the matrix of parameters may be a representation of method steps, e.g. a list thereof, comprising the aforesaid parameters and/or parameter values. Also in an embodiment, the matrix of parameters comprises further information, in particular on the result of a comparison of the matrix of parameters to a reference matrix of parameters, wherein, in an embodiment, a reference matrix of parameters is a matrix of predefined parameters and/or values thereof, such as of intended parameters and/or values thereof; or, in a further embodiment, the reference matrix of parameters is a representation of method steps, e.g. a list thereof, comprising the aforesaid parameters and/or parameter values, of a desired method, of a previously used method, or of a comparison method. Thus, in an embodiment, the matrix of parameters comprises a comparison of said matrix of parameters to a reference matrix of parameters, wherein said comparison may e.g. be provided by highlighting differences between the matrix of parameters and the reference matrix of parameters, or by printing, in case a difference is identified, by displaying the parameter and/or parameter value from the matrix of parameters and of the reference matrix of parameters. Also in an embodiment, such identified difference is applied to an automatic plausibility check as specified elsewhere herein.

Also in an embodiment, step iii. may be automatically displaying on a display a representation of method steps comprising further information, in particular on the result of a comparison of method steps to a reference list of method steps, wherein, in an embodiment, a reference list of method steps is a matrix of predefined method steps, parameters, and/or values thereof, such as of intended parameters and/or values thereof; i.e., in a further embodiment, the reference list of method steps is a representation of method steps, e.g. a list thereof, comprising the aforesaid parameters and/or parameter values, of a desired method, of a previously used method, or of a comparison method. Thus, in an embodiment, the list of method steps comprises a comparison of said list of method steps to a reference list of method steps, wherein said comparison may e.g. be provided by highlighting differences between the list of method steps and the reference list of method steps, or by printing, in case a difference is identified, by displaying the parameter and/or parameter value from the list of method steps and of the reference list of method steps. Also in an embodiment, such identified difference is applied to an automatic plausibility check as specified elsewhere herein.

The parameters of interest of the program cycles may be combined in a combined line of the matrix if the parameters of interest of these program cycles have identical parameter values.

The parameters of interest may be identical for at least a fraction of the program cycles controlling the elongation cycles, specifically for all program cycles controlling elongation cycles, more specifically for all program cycles, such that the matrix comprises the parameter values for identical parameters of different program cycles of the program code.

The method may further comprise:
iii. automatically displaying the matrix of parameters on a display.

The term “displaying” as used herein is a broad term and is to be given its ordinary and customary meaning to a person of ordinary skill in the art and is not to be limited to a special or customized meaning. The term specifically may refer, without limitation, to a process of representing information in a visual form. Consequently, the term “display” may refer, without limitation, to an illustrating user interface configured for representing information in a visual form. In particular, the display device may comprise at least one screen. For example, the screen may have a flat and/or even surface. As an example, the display may be or may comprise a liquid-crystal display (LCD), such as a flat-panel display, e.g. an electronically modulated optical device, using light-modulating properties of liquid crystals. Other types of displays may be possible, such as light-emitting diode (LED) displays or the like. Displaying may, however, also be outputting information on a permanent medium, e.g. by printing.

The method may further comprise:
iv. applying an automatic error detection step to the matrix of parameters, the automatic error detection step comprising subjecting the matrix of parameters to at least one automatic plausibility check.

The term “error detection step” as used herein is a broad term and is to be given its ordinary and customary meaning to a person of ordinary skill in the art and is not to be limited to a special or customized meaning. The term specifically may refer, without limitation, to a process of determining deviations of an item of information compared with a reference item of information. Specifically, the error detection step may comprise determining deviations in the matrix of parameters from a reference matrix of parameters indicating the parameters and/or the corresponding parameter values for a given synthesis of the oligonucleotide. Alternatively or additionally, the error correction step may comprise detecting a deviation of a parameter value from an optimal value in the program code. Thus, the error may comprise a deviation of a parameter value from an optimal value, such as a value being optimal for the synthesis of the oligonucleotide.

The term “plausibility check” as used herein is a broad term and is to be given its ordinary and customary meaning to a person of ordinary skill in the art and is not to be limited to a special or customized meaning. The term specifically may refer, without limitation, to a process of classifying the matrix of parameters in binary classes, e.g. “plausible” and “not plausible”. For example, the plausibility check may comprise classifying the parameter values comprised by the matrix of parameters. The automatic plausibility check may comprise classifying the parameter value as “plausible” if the parameter value is equal to or lies within a predetermined and/or determinable value range. The automatic plausibility check may comprise classifying the parameter value as “not plausible” if the parameter value lies outside the predetermined and/or determinable value range. The value range may be defined in absolute values, such as by defining upper and/or lower boundaries. Alternatively or additionally, the value range may be defined relatively to a target value of the parameter value, such as by defining a tolerance range with regard to the target value of the parameter value. For example, a parameter value lies within the value range if the parameter value deviates by no more than 10%, specifically by no more than 5%, more specifically by no more than 1%, from the target value of the parameter value. Alternatively or additionally, the automatic plausibility check may comprise classifying a pattern of the matrix of parameters.

The automatic plausibility check may specifically comprise at least one of the following:
comparing a pattern of the matrix of parameters with at least one predetermined target pattern;
detecting missing parameter values in the matrix of parameters;
detecting incorrect units;
detecting parameter values out of a predetermined range;
detecting missing lines in the matrix;
detecting additional lines in the matrix;
identifying parameters of interest of the program cycles being listed in different lines of the matrix which should be listed in a combined line of the matrix;
detecting the selection of correct reagents;
detecting the selection of correct solvents;
detecting the selection of correct starting materials.

Further, the automatic error detection step may comprise an automatic output of warning information to a user if the automatic error detection step detects an error in the program code.

Additionally or alternatively, the automatic error detection step may comprise an automatic prevention of the synthesis computer program from being executed on said automatic synthesizer if the automatic error detection step detects an error in the program code. The warning information may specifically comprise information on at least one of: a type of the error in the program code; a position of the error in the program code; an identification of the program cycle in which the error occurred; an identification of the parameter of interest in which the error occurred.

As outlined above, the automatic error detection step may also comprise an automatic prevention of the synthesis computer program from being executed on the automatic synthesizer. Thus, the detection of an error may cause the automatic generation of a signal bringing the automatic synthesizer into a state in which the synthesis computer program cannot be automatically executed. Said state may allow a manual override by the user, e.g. after manual error correction, or may allow execution of the synthesis computer program only after the method of verifying a program code was performed again, in an embodiment without detecting an error.

In the automatic error detection step, at least one of the following errors may be detected: incorrect units of the parameters; an omitted setting of at least one chemical, specifically of at least one reactant and/or at least one solvent; an incorrect setting of at least one valve; a flow of at least one chemical, specifically of at least one reactant and/or of at least one solvent, not being set to zero at the end of the program code.

The method may further comprise:
v. an automatic error correction step, the automatic error correction step comprising automatically correcting at least one error in the program code detected in the automatic error detection step.

The term “error correction step” as used herein is a broad term and is to be given its ordinary and customary meaning to a person of ordinary skill in the art and is not to be limited to a special or customized meaning. The term specifically may refer, without limitation, to a process of adjusting the program code. The automatic error correction step may comprise adjusting the program code, specifically the instructions in the program code, such that the resulting matrix of parameters passes the automatic plausibility check, e.g. by being classified as “plausible”. Thus, the automatic error correction step may comprise identifying the instructions in the program code associated with deviations in the matrix of parameters and, further, replacing the identified instructions by corrected instructions. The automatic error correction step may specifically be performed automatically as defined above, in particular such that the automatic error correction step is at least partially performed by means of at least one computer, in particular without manual action and/or interaction with a user. Alternatively or additionally, the error correction step may comprise adjusting the program code by optimizing a detected deviation of a parameter value in the program code. Specifically, in case a deviation of a parameter value from an optimal value may be detected in the automatic error detection step, the error correction step may comprise optimizing the parameter value, specifically by minimizing the deviation of the parameter value compared with the optimal value.

Also in an embodiment, the method may further comprise:
vi. automatically calculating a total required amount of at least one reagent based on said parameter values.

The term “required amount”, as used herein, is a broad term and is to be given its ordinary and customary meaning to a person of ordinary skill in the art and is not to be limited to a special or customized meaning. The term specifically may refer to an amount of reagent necessary to complete a predetermined number of program cycles as specified herein. In accordance, a “total required amount”, as referred to herein, relates to the amount of reagent necessary to complete a synthesis controlled by a synthesis computer program, i.e., e.g., to synthesize an oligonucleotide. As is understood by the skilled person, an amount of reagent may be calculated as a volume, as a weight or mass, as an amount of substance, or the like. Also in an embodiment, the calculated total required amount may be presented to an operator of the automatic synthesizer, e.g. to ensure that sufficient reagent is prepared for a planned synthesis; or the calculated total required amount may be interfaced, in an embodiment automatically, to an automated pipetting device.

The method may specifically be computer-implemented. The term “computer-implemented” as used herein, specifically in the context of the method, is a broad term and is to be given its ordinary and customary meaning to a person of ordinary skill in the art and is not to be limited to a special or customized meaning. The term specifically may refer, without limitation, to a characteristic of the method of involving at least one computer. The computer may comprise at least one processor which is configured for performing at least one of the method steps of the method according to the present invention. Preferably, each of the method steps may be performed by the computer. The method may be performed completely automatically, specifically without user interaction.

As will be understood from the specifications provided herein, the method of verifying a program code may be implemented on a computer being part of an automatic synthesizer. The method of verifying a program code may, however, also be computer-implemented on an independent computer, which may be connected to an automatic synthesizer via a data transfer means.

In a further aspect of the present invention, a computer program is disclosed, comprising instructions which, when the program is executed by a computer or a computer system, cause the computer or computer system to perform the method according to the present invention, specifically the method of verifying a program code of a synthesis computer program according to the present invention, such as according to any one of the embodiments disclosed above and/or according to any one of the embodiments disclosed in further detail below.

For definitions and embodiments of the computer program, reference is made to the definitions and embodiments of the method of verifying a program code of a synthesis computer program as described above.

In a further aspect of the present invention, a computer-readable storage medium, specifically a non-transient computer-readable storage medium, is disclosed, comprising instructions which, when the instructions are executed by a computer or a computer system, cause the computer or computer system to perform the method according to the present invention, specifically the method of verifying a program code of a synthesis computer program according to the present invention, such as according to any one of the embodiments disclosed above and/or according to any one of the embodiments disclosed in further detail below.

For definitions and embodiments of the computer-readable storage medium, reference is made to the definitions and embodiments of the method of verifying a program code of a synthesis computer program as described above.

As used herein, the term “computer-readable storage medium” specifically may refer to non-transitory data storage means, such as a hardware storage medium having stored thereon computer-executable instructions. The computer-readable storage medium specifically may be or may comprise a storage medium such as a random-access memory (RAM) and/or a read-only memory (ROM). Consequently, the term “non-transient” as used herein may refer, without limitations, to a property of the computer-readable storage medium of being configured for permanently storing the computer-executable instructions. Thus, this property of the computer-readable storage medium may also be referred to as “non-transitory”.

In a further aspect of the present invention, a system for verifying a program code of a synthesis computer program is disclosed. The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components forming a whole. Specifically, the components may interact with each other in order to fulfill at least one common function. The at least two components may be handled independently or may be coupled or connectable.

The system comprises at least one processor, as defined above. The processor is configured by programming, specifically by software programming, for performing the method according to the present invention, specifically the method of verifying a program code of a synthesis computer program according to the present invention, such as according to any one of the embodiments disclosed above and/or according to any one of the embodiments disclosed in further detail below.

For further definitions and embodiments of the system, reference is made to the definitions and embodiments of the method of verifying a program code of a synthesis computer program as described above.

In a further aspect of the present invention, a method of synthesizing at least one oligonucleotide is disclosed.

The method of synthesizing at least one oligonucleotide comprises the following steps which, as an example, may be performed in the given order. It shall be noted, however, that a different order is also possible. Further, it is also possible to perform one, more than one or even all of the method steps once or repeatedly. Further, it is possible to perform two or more of the method steps simultaneously or in a timely overlapping fashion. The method may comprise further method steps which are not listed.

The method of synthesizing at least one oligonucleotide comprises:
a. providing a program code of a synthesis computer program, the synthesis computer program being configured for computer-controlling at least one automatic synthesizer, the synthesis computer program having a plurality of program cycles for computer-controlling the automatic synthesizer to sequentially synthesize the oligonucleotide by using a sequence of synthesis cycles;
b. applying the method of verifying the program code according to the present invention, such as according to any one of the embodiments disclosed above and/or according to any one of the embodiments disclosed in further detail below; and
c. computer-controlling at least one an automatic synthesizer to automatically synthesize the at least one oligonucleotide.

For definitions and embodiments of the method of synthesizing at least one oligonucleotide, reference is made to the definitions and embodiments of the method of verifying a program code of a synthesis computer program as described above.

The method may further comprise, before step c.:
d. at least one correction step, comprising amending at least one parameter value of at least one parameter of interest in accordance with at least one result of step b.

In step c., at least one automatic synthesizer may be used. Embodiments of automatic synthesizers have been described herein above. Step c. may comprise computer-controlling at least the switchable valves and the at least one pump and/or additional units optionally present in the automatic synthesizer as specified herein above. In an embodiment, step c. comprises performing at least one initiation cycle and at least one elongation cycle; step c. may, however, also comprise performing at least two elongation cycles, e.g. in case the method does not require attachment of a first nucleoside to a solid support or if the solid matrix comprises a suitable attachment moiety.

Furthermore, the present invention also relates to a method of verifying a program code of a synthesis computer program, the synthesis computer program being configured for computer-controlling an automatic synthesizer to automatically synthesize at least one biological macromolecule, the synthesis computer program having a plurality of program cycles for computer-controlling the automatic synthesizer to sequentially synthesize the biological macromolecule by using at least one sequence of synthesis cycles, the method comprising:
i. applying an automatic parsing procedure to the program code of the synthesis computer program, the automatic parsing procedure comprising automatically searching for parameter values of parameters of at least one predetermined list of parameters of interest in the program cycles of the synthesis computer program; and
ii. automatically assembling a matrix of parameters comprising, for the program cycles of the synthesis computer program, the parameters of interest and the corresponding parameter values of the parameters of interest.

With respect to the method of verifying a program code of a synthesis computer program, the synthesis computer program being configured for computer-controlling an automatic synthesizer to automatically synthesize at least one biological macromolecule, the definitions and embodiments described herein above for the method of verifying a program code of a synthesis computer program, the synthesis computer program being configured for computer-controlling an automatic synthesizer to automatically synthesize at least one oligonucleotide apply mutatis mutandis.

The term “biological macromolecule” as used herein is a broad term and is to be given its ordinary and customary meaning to a person of ordinary skill in the art and is not to be limited to a special or customized meaning. The term specifically may refer, without limitation, to any molecule comprising, in an embodiment consisting of, a multitude of units of which, in an embodiment, at least two are non-identical. In an embodiment, the biological macromolecule is synthesized chemically in the automatic synthesizer, i.e. in an embodiment is not synthesized in a biological system such as a cell or an in vitro transcription and/or translation system. In an embodiment, the biological macromolecule is synthesized by solid phase synthesis. The biological macromolecule may be an oligonucleotide or a peptide, in an embodiment is an oligonucleotide, in a further embodiment is a peptide.

The term “peptide” as used herein is a broad term and is to be given its ordinary and customary meaning to a person of ordinary skill in the art and is not to be limited to a special or customized meaning. The term specifically may refer, without limitation, to any molecule comprising at least one peptide bond. In an embodiment, the peptide comprises of from 2 to 100 amino acid units, in an embodiment of from 3 to 50 amino acid units, in a further embodiment of from 4 to 25 amino acid units. In an embodiment, the amino acid units are alpha-amino acids, in a further embodiment are L-alpha-amino acids, although modified amino acids and/or D-amino acids and/or non-alpha amino acids may be used as well. Methods of synthesizing peptides are known in the art; reference is e.g. made to the Wikipedia entry for “peptide synthesis” as of 24 May 2022.

The methods, the computer program, the computer-readable storage medium and the system according to the present invention provide a large number of advantages over known methods and devices of similar kind. Specifically, the methods, the computer program, the computer-readable storage medium and the system may provide means for verifying the program code of the synthesis program for synthesizing the at least one oligonucleotide. Errors in the program code may be easily detected and may be brought to the user's attention.

Thus, as an example, the method of verifying the program code, which specifically may be a computer-implemented method, may use the program code of the synthesis computer program as an input and may, specifically fully or at least partially automatically, generate, from the input, an output comprising the matrix of parameters including the parameters of interest and the corresponding parameter values of the parameters of interest. Consequently, no matter how complex the synthesis computer program and the corresponding program code may be, including numerous parameters and program cycles, the method may automatically generate from the program code an item of information in the form of the matrix of parameters which may easily be checked, either by a user or even automatically, for plausibility. The method, thus, automatically reduces the complexity of the program code of the synthesis computer program, to the matrix of parameters comprising the parameters of interest. An evaluation of the correctness of the synthesis computer program and a corresponding program code may, thus, be reduced to an evaluation of the correctness of the matrix of parameters. Thereby, the efficiency of the verification of the computer program, by a human user or automatically, as well as the reliability of the verification may be greatly increased. Instead of checking the entire and often complex program code, including its many iterations, routines and subroutines, for the presence of specific parameters and/or the correctness of the parameter values of the specific parameters, the method may reduce the work to checking the matrix of parameters. The matrix of parameters may be visualized for control by a human being and/or may be subject to the at least one automatic error detection step, which, as compared to an error detection in the “raw” program code, is greatly simplified and requires significantly less resources.

Specifically, the matrix of parameter may provide a clear and compact visualization of the parameter and corresponding parameter values at the automatic synthesizer. The program code itself or user interface are generally complex and unclear. Thus, errors in the program code can be easily detected and corrected, either manually or automatically. Therefore, the methods, the computer program, the computer-readable storage medium and the system may be suitable for providing robust and reliable automatic synthesis of oligonucleotides.

Further, the method of verifying a program code of a synthesis computer program may allow going sequentially through all steps of the procedure, specifically through all of the instructions comprised by the program code, and keeping track of changes in reagents, solvent flow rates, volumes and/or times. At the end of each operation, for example when a flow is stopped, the parameter values of the parameters of interest may be summarized in the matrix of parameters, such as reagents, amounts, flow rates and time, for the two or more pumps of the automatic synthesizer. The assembled matrix of parameters may group all the parameter values for each operation by cycle numbers and, thus, can be presented and displayed in a easy and comprehensive manner to a user. Further, the matrix of parameters can be compared with a given synthesis plan and/or with matrices of parameters in order to detect errors in the program code. Graphical visualizations may be used to easily detect deviations in values and/or deviations in patterns.

Even though generally also applicable to other synthesis computer programs, configured for computer-controlling other types of syntheses than the automatic synthesis of oligonucleotides, the method is specifically advantageous for the synthesis of oligonucleotides. This is specifically due to the fact that the synthesis of oligonucleotides typically comprises, as indicated above, a sequence of synthesis cycles of identical or similar type. In each cycle, as an example, a nucleotide may be added to a growing sequence, thereby sequentially building up or synthesizing the desired oligonucleotide. In this case, even though the chemical process generally comprises a plurality of synthesis cycles of similar or even identical chemical nature, the synthesis computer program and the corresponding program code may be rather complex. The method as proposed herein, however, greatly reduces the efforts required for the verification of the program code, since the predetermined list of parameters of interest may reflect the determining parameters of the program cycles. Consequently, the iterative nature of the chemical process of the synthesis of the oligonucleotides may be used for reducing the complexity of the verification of the program code of the corresponding synthesis computer program. Further, the method of verifying a program code of a synthesis computer program may also be used to automatically fill gaps in the program code from the matrix of parameters, such as to complete an incomplete program code. This may additionally reduce human resource requirements and reduce errors.

Also in an embodiment, the present invention relates to a method of verifying a set of at least two program codes of synthesis computer programs, each synthesis computer program being configured for computer-controlling an automatic synthesizer (114) to automatically synthesize at least one oligonucleotide, each synthesis computer program having a plurality of program cycles for computer-controlling the automatic synthesizer (114) to sequentially synthesize the oligonucleotide by using at least one sequence of synthesis cycles, the method comprising:
(I) applying an automatic parsing procedure to the program code of each of said synthesis computer programs, the automatic parsing procedure comprising automatically searching for parameter values of parameters of at least one predetermined list of parameters of interest in the program cycles of the synthesis computer programs;
(II) automatically assembling a verification matrix of parameters comprising, for the program cycles of each of the synthesis computer programs, (i) the parameters of interest and the corresponding parameter scores of the parameters of interest, or (ii) a structured array of parameter scores of said parameters of interest; and
(III) comparing a pattern of the verification matrix of parameters with at least one predetermined target pattern.

The method of verifying a set of at least two program codes of the present invention may comprise steps in addition to those explicitly mentioned above. For example, further steps may relate, e.g., to automatically displaying a list of synthesis computer programs for which a deviation from a predetermined target pattern was detected in step (III).

The term “verification matrix of parameters”, as used herein, is a broad term and is to be given its ordinary and customary meaning to a person of ordinary skill in the art and is not to be limited to a special or customized meaning. The term specifically may refer to a matrix of parameters as specified herein above comprising parameters and/or parameters scores of at least two program codes of synthesis computer programs, in an embodiment of all program codes of synthesis computer programs of a given set. As the skilled person understands in view of the description herein, the verification matrix of parameters may e.g. comprise at least one parameter allocated to the values of said parameter in the synthesis computer programs, in an embodiment in a structured array; or may comprise a matrix of parameters as specified herein above for each of said synthesis computer programs; or may comprise a structured array of parameter scores of said parameters, as specified herein below.

The term “parameter score”, as used herein, is a broad term and is to be given its ordinary and customary meaning to a person of ordinary skill in the art and is not to be limited to a special or customized meaning. The term specifically may refer to any representation of a parameter value or of a range of parameter values; such representation may be e.g. a symbol, a number which does not necessarily have to correspond to the parameter value, a letter, or the like, in particular in case the result of step (II) is output to a user; or it may be any internal state of a microprocessor and/or of a data storage device enabling unambigiuos allocation of parameter scores and comparison thereof. In an embodiment, the parameter score is a parameter value.

The term “structured array of parameter scores”, as used herein, is a broad term and is to be given its ordinary and customary meaning to a person of ordinary skill in the art and is not to be limited to a special or customized meaning. The term specifically may refer to an array of parameter scores in which the score of a given parameter is placed at a predetermined position within said array. Thus, in a structured array of parameter scores, each parameter score can be unambiguously allocated to its corresponding parameter based on its position within the array. Thus, in an embodiment, the structured array of parameter scores is a shorthand display of parameter/parameter score data.

The term “verifying a set of at least two program codes”, as used herein, is a broad term and is to be given its ordinary and customary meaning to a person of ordinary skill in the art and is not to be limited to a special or customized meaning. The term specifically may refer to verifying steps and/or parameters of a set of program codes, in an embodiment to verifying parameters of a set of program codes. Thus, in an embodiment, the program codes of the set of program codes are compared to each other and/or to a reference program code, and a verification matrix of parameters of interest is established; such a verification matrix of parameters may comprise parameters of interest and the corresponding parameter scores of the parameters of interest, e.g. in the form of a list or as a table; or said verification matrix of parameters may comprise a structured array of parameter scores of said parameters of interest, as specified herein above. Thus, step (III) may comprise, in an embodiment automatically, comparing the verification matrix of parameters obtained in step (II) to a target pattern, e.g. a set of target matrices of parameters, or to target structured arrays. In an embodiment, step (III) comprises comparing a list of structured arrays obtained in step (II) to a target list of structured arrays. In an embodiment, the target pattern is a combinatorial design of experiments pattern. In an embodiment, the comparing in step (III) is part of an automatic plausibility check as specified elsewhere herein. Also in an embodiment, in case a difference is identified between the verification matrix of parameters and the predetermined target pattern, execution of said set of at least two program codes of synthesis computer programs is automatically prevented or stopped and/or a warning is displayed. Summarizing and without excluding further possible embodiments, the following embodiments may be envisaged:

Embodiment 1: A method of verifying a program code of a synthesis computer program, the synthesis computer program being configured for computer-controlling an automatic synthesizer to automatically synthesize at least one oligonucleotide, the synthesis computer program having a plurality of program cycles for computer-controlling the automatic synthesizer to sequentially synthesize the oligonucleotide by using at least one sequence of synthesis cycles, the method comprising:
i. applying an automatic parsing procedure to the program code of the synthesis computer program, the automatic parsing procedure comprising automatically searching for parameter values of parameters of at least one predetermined list of parameters of interest in the program cycles of the synthesis computer program; and
ii. automatically assembling a matrix of parameters comprising, for the program cycles of the synthesis computer program, the parameters of interest and the corresponding parameter values of the parameters of interest.

Embodiment 2: The method according to the preceding embodiment, wherein said synthesis cycles comprise at least two elongation cycles wherein a nucleotide residue is added to the oligonucleotide chain.

Embodiment 3: The method according to the preceding embodiment, wherein the parameters of interest are identical for at least a fraction of the program cycles controlling the elongation cycles, specifically for all program cycles controlling elongation cycles, more specifically for all program cycles, such that the matrix comprises the parameter values for identical parameters of different program cycles of the program code.

Embodiment 4: The method according to any one of the preceding embodiments, wherein, in the automatic parsing procedure, a list of keywords is assembled corresponding to the list of parameters of interest, the keywords of the list of keywords being known to be used in conjunction with the corresponding parameters of interest of the list of parameters of interest in the program code of the synthesis computer program, wherein the parameter values of the parameters of interest are known to be located in the program code in a predetermined relative location relative to the keywords, wherein the automatic parsing procedure further comprises reading the corresponding parameter values of the parameters of interest from the predetermined relative location of the corresponding keywords.

Embodiment 5: The method according to any one of the preceding embodiments, the method further comprising:
iii. automatically displaying the matrix of parameters on a display.

Embodiment 6: The method according to any one of the preceding embodiments, wherein parameters of interest of the program cycles are combined in a combined line of the matrix if the parameters of interest of these program cycles have identical parameter values.

Embodiment 7: The method according to any one of the preceding embodiments, wherein the predetermined list of parameters of interest comprises at least one parameter of interest selected from the group consisting of: a type of a chemical, specifically a reactant and/or a solvent; a volume of a chemical, specifically a reactant and/or a solvent; a flow rate of a chemical, specifically of a reactant and/or of a solvent; a temperature of a chemical, specifically of a reactant and/or of a solvent; a pump pressure of at least one pump or the length of a particular synthesis step, e.g. the length of a recycling step at a certain flow rate X for Y minutes.

Embodiment 8: The method according to any one of the preceding embodiments, the method further comprising:
iv. applying an automatic error detection step to the matrix of parameters, the automatic error detection step comprising subjecting the matrix of parameters to at least one automatic plausibility check.

Embodiment 9: The method according to the preceding embodiment, wherein the automatic plausibility check comprises at least one of the following:
comparing a pattern of the matrix of parameters with at least one predetermined target pattern;
detecting missing parameter values in the matrix of parameters;
detecting incorrect units;
detecting parameter values out of a predetermined range;
detecting missing lines in the matrix;
detecting additional lines in the matrix;
identifying parameters of interest of the program cycles being listed in different lines of the matrix which should be listed in a combined line of the matrix;
detecting the selection of correct reagents;
detecting the selection of correct solvents;
detecting the selection of correct starting materials.

Embodiment 10: The method according to any one of the two preceding embodiments, further comprising (I) an automatic output of warning information to a user and/or (II) an automatic prevention of the synthesis computer program from being executed on said automatic synthesizer (114) if the automatic error detection step detects an error in the program code.

Embodiment 11: The method according to the preceding embodiment, wherein the warning information comprises information on at least one of: a type of the error in the program code; a position of the error in the program code; an identification of the program cycle in which the error occurred; an identification of the parameter of interest in which the error occurred.

Embodiment 12: The method according to any one of the four preceding embodiments, wherein, in the automatic error detection step, at least one of the following errors is detected: incorrect units of the parameters; an omitted setting of at least one chemical, specifically of at least one reactant and/or at least one solvent; an incorrect setting of at least one valve; a flow of at least one chemical, specifically of at least one reactant and/or of at least one solvent, not being set to zero at the end of the program code.

Embodiment 13: The method according to any one of the five preceding embodiments, further comprising:
v. an automatic error correction step, the automatic error correction step comprising automatically correcting at least one error in the program code detected in the automatic error detection step.

Embodiment 14: The method according to any one of the preceding embodiments, wherein the matrix of parameters comprises a comparison of said matrix of parameters to a reference matrix of parameters, optionally by highlighting differences between the matrix of parameters and the reference matrix of parameters.

Embodiment 15: The method according to any one of the preceding embodiments, wherein step iii. is automatically displaying on a display a representation of method steps comprising a comparison of method steps to a reference list of method steps.

Embodiment 16: The method according to any one of the preceding embodiments, further comprising:
vi. automatically calculating a total required amount of at least one reagent based on said parameter values.

Embodiment 17: A method of verifying a set of at least two program codes of synthesis computer programs, each synthesis computer program being configured for computer-controlling an automatic synthesizer (114) to automatically synthesize at least one oligonucleotide, each synthesis computer program having a plurality of program cycles for computer-controlling the automatic synthesizer (114) to sequentially synthesize the oligonucleotide by using at least one sequence of synthesis cycles, the method comprising:
(I) applying an automatic parsing procedure to the program code of each of said synthesis computer programs, the automatic parsing procedure comprising automatically searching for parameter values of parameters of at least one predetermined list of parameters of interest in the program cycles of the synthesis computer programs;
(II) automatically assembling a verification matrix of parameters comprising, for the program cycles of each of the synthesis computer programs, (i) the parameters of interest and the corresponding parameter scores of the parameters of interest, or (ii) a structured array of parameter scores of said parameters of interest; and
(III) comparing a pattern of the verification matrix of parameters with at least one predetermined target pattern.

Embodiment 18: The method according to any one of the preceding embodiments, wherein the program code is a method file, specifically a Unicorn method file.

Embodiment 19: The method according to any one of the preceding embodiments, wherein the program code is a binary program code.

Embodiment 20: The method according to any one of the preceding embodiments, wherein the program code comprises a header and a text section following the header, wherein the method comprises an automatic detection of the text section in the program code, wherein the automatic parsing procedure of step i. is performed on the text section.

Embodiment 21: The method according to anyone of the preceding method embodiments, wherein the method is computer-implemented.

Embodiment 22: A computer program comprising instructions which, when the program is executed by a computer or a computer system, cause the computer or computer system to perform the method according to any one of the preceding embodiments referring to a method.

Embodiment 23: A computer-readable storage medium, specifically a non-transient computer-readable storage medium, comprising instructions which, when the instructions are executed by a computer or a computer system, cause the computer or computer system to perform the method according to any one of the preceding embodiments referring to a method.

Embodiment 24: A system for verifying a program code of a synthesis computer program, the system comprising at least one processor, the processor being configured by programming, for performing the method according to any one of the preceding embodiments referring to a method.

Embodiment 25: A method of synthesizing at least one oligonucleotide, the method comprising:
a. providing a program code of a synthesis computer program, the synthesis computer program being configured for computer-controlling at least one automatic synthesizer, the synthesis computer program having a plurality of program cycles for computer-controlling the automatic synthesizer to sequentially synthesize the oligonucleotide by using a sequence of synthesis cycles;
b. applying the method of verifying the program code according to any one of the preceding embodiments referring to a method; and
c. computer-controlling at least one an automatic synthesizer to automatically synthesize the at least one oligonucleotide.

Embodiment 26: The method according to the preceding embodiment, the method further comprising, before step c.:
d. at least one correction step, comprising amending at least one parameter value of at least one parameter of interest in accordance with at least one result of step b.

Embodiment 27: The method of synthesizing according to any one of the preceding embodiments referring to a method of synthesizing, wherein the at least one oligonucleotide comprises at least one oligonucleotide selected from the group consisting of: a DNA oligonucleotide, an RNA oligonucleotide, an LNA oligonucleotide, specifically, wherein said oligonucleotide comprises at least two, more specifically at least three, even more specifically at least four non-identical nucleotides.

Embodiment 28: The method of synthesizing according to any one of the preceding embodiments referring to a method of synthesizing, wherein, in step c., at least one automatic synthesizer is used, the automatic synthesizer comprising at least one synthesis column having at least one stationary phase material, the automatic synthesizer further comprising at least one pump, connectors to a plurality of chemical supplies and a system of switchable valves for selectively supplying chemicals, specifically solvent and/or reactants, to the column, wherein step c. comprises computer-controlling at least the switchable valves and the at least one pump.

Embodiment 29: The method according to the preceding embodiment, wherein the automatic synthesizer further comprises at least one further element, wherein step c. comprises computer-controlling the at least one further element.

Embodiment 30: The method according to the preceding embodiment, wherein the further element is selected from the group consisting of an amidite pump; an amidite valve; a solvent/reagent pump; a solvent/reagent valve; a system of switchable valves for feeding liquid from the column into predetermined receptacles; at least one tempering element for setting the column to at least one predetermined temperature; a column inlet valve for admitting liquid flow into the column; a column outlet valve for admitting liquid flow from the column; a recycling bypass, specifically for recycling liquid over the column, such as by connecting a column outlet valve to a column inlet valve by means of at least one valve; a waste valve for directing liquid from the column into at least one waste system.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an embodiment of a system for verifying a program code of a synthesis computer program, and an automatic synthesizer, in a schematic view;

FIG. 2 shows a flow chart of an embodiment of a method of verifying a program code of a synthesis computer program; and

FIG. 3 shows a flow chart of an embodiment of a method of synthesizing at least one oligonucleotide.

### DETAILED DESCRIPTION OF THE EMBODIMENTS

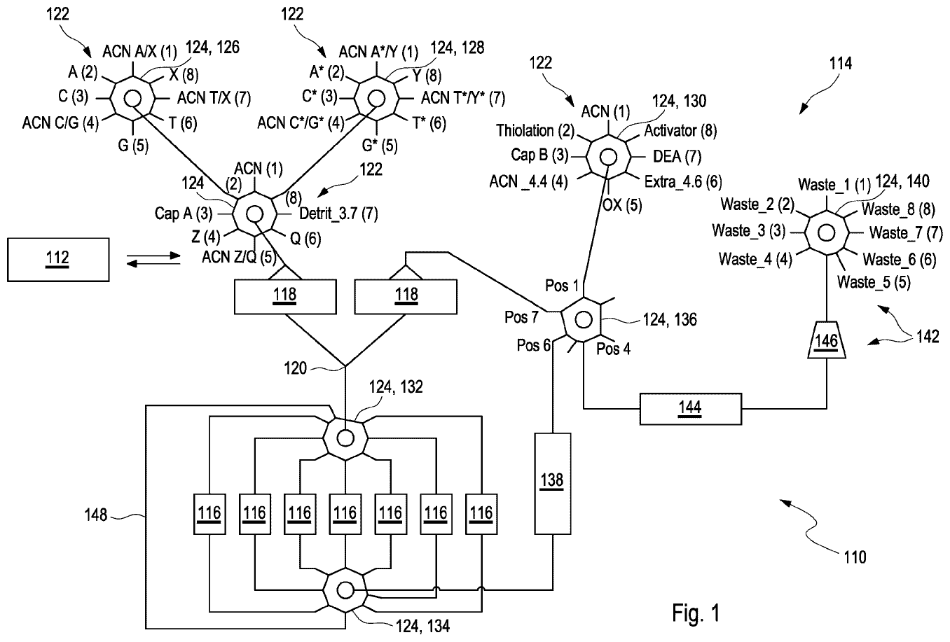

FIG. 1 shows an exemplary embodiment of a system 110 for verifying a program code of a synthesis computer program and an automatic synthesizer 114 in a schematic view. The system 110 may be combined, or coupled, as shown in FIG. 1, with the automatic synthesizer 114 or may be separate components. The automatic synthesizer 114 shown in FIG. 1 may be a oligoplot synthesizer which is commercially available by the company Cytiva®. However, other types of automatic synthesizer 144 are also feasible.

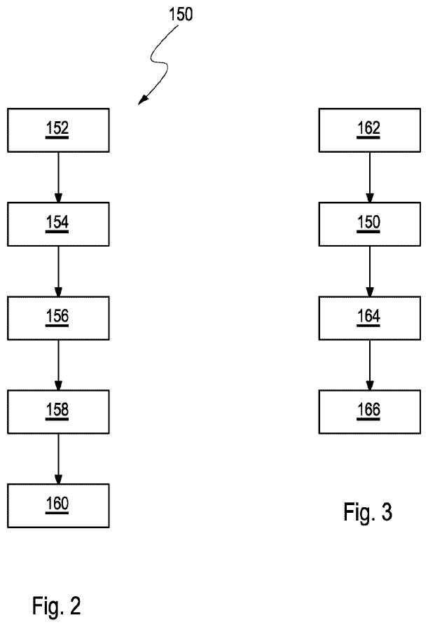

The system 110 comprises at least one processor 112. The processor 112 is configured by programming, specifically by software programming, for performing a method of verifying a program code of a synthesis computer program according to the present invention, such as according to the exemplary embodiment shown in FIG. 2 and/or according to any other embodiment disclosed herein. Thus, for a description of the method of verifying a program code of a synthesis computer program, reference is made to the description of FIG. 2.

The system 110 may further comprise at least one automatic synthesizer 114 or may interact with at least one automatic synthesizer 114, such that a program code of a synthesis computer program verified by the system 110 may then be executed by the automatic synthesizer 114. For this purpose, the system 110 and the automatic synthesizer 114 may be fully or partially integrated into one another, may be connected by at least one interface or network, or the verified program code may be transferred to the automatic synthesizer 114 by other means, such as by a portable data storage device and/or by wireless or wirebound data transfer.

The automatic synthesizer 114 may comprise at least one synthesis column 116 having at least one stationary phase material. Specifically, as can be seen in FIG. 1, the automatic synthesizer 114 may comprise a plurality of columns 116, e.g. seven columns 116 or even more.

The automatic synthesizer 114 may further comprise at least one pump 118, connectors 120 to a plurality of chemical supplies 122 and a system of switchable valves 124 for selectively supplying chemicals, specifically solvent and/or reactants, to the columns 116. Specifically, in the exemplary system 110 shown in FIG. 1, the automatic synthesizer 114 may comprise amidite valves 126, 128. The amidite valves 126, 128 may provide liquid via a further valve 124 to the pump 118.

The automatic synthesizer 114 may further comprise a reagent valve 130. Additionally, the automatic synthesizer 114 may comprise a column inlet valve 132 for admitting liquid flow into the column 116 and a column outlet valve 134 for admitting liquid flow from the column 116. As can be seen in FIG. 1, the column inlet valve 132 may be arranged downstream the connector 120 and upstream the columns 116. The column outlet valve 134 may be arranged downstream the columns 116 and may connect liquid flowing from the columns 116 to a recycle valve 136. In between the column outlet valve 132 and the recycle valve 136, the automatic synthesizer 114 may comprise a pH-meter, a conductivity sensor and/or a combined pH-C-meter 138 for monitoring liquid flowing from the columns 116. Thus, the recycle valve 136 may configured for recycling liquid flowing from the columns 116 and/or to dispense liquid via a waste valve 140 for directing liquid from the columns 116 into at least one waste system 142. As an example, the waste system 142 may comprise an UV-absorption sensor 144 and a flow restrictor 146.

The automatic synthesizer 114 may further comprise a column bypass 148 for providing bypass flow from the column inlet valve 132 to the column outlet valve 134.

The automatic synthesizer 114 may comprise further elements, such as amidite pumps, solvent/reagent pumps and/or tempering elements for setting the columns 116 to at least one predetermined temperature (not shown in FIG. 1).

Thus, the system 110, in the configuration shown in FIG. 1, may be configured for performing a method of synthesizing at least one oligonucleotide according to the present invention, such as according to the exemplary embodiment shown in FIG. 3 and/or according to any other embodiment disclosed herein. Thus, for a description of the method of synthesizing at least one oligonucleotide, reference is made to the description of FIG. 3.

FIG. 2 shows a flow chart of an exemplary embodiment of a method of verifying a program code of a synthesis computer program. In the Figures, the method of verifying a program code of a synthesis computer program is denoted by reference number 150. The synthesis computer program is configured for computer-controlling the automatic synthesizer 114 to automatically synthesize at least one oligonucleotide, the synthesis computer program having a plurality of program cycles for computer-controlling the automatic synthesizer 114 to sequentially synthesize the oligonucleotide by using at least one sequence of synthesis cycles.

The method comprises the following steps which, as an example, may be performed in the given order. It shall be noted, however, that a different order is also possible. Further, it is also possible to perform one, more than one or even all of the method steps once or repeatedly. Further, it is possible to perform two or more of the method steps simultaneously or in a timely overlapping fashion. The method may comprise further method steps which are not listed.

The method comprises:
i. (denoted by reference number 152) applying an automatic parsing procedure to the program code of the synthesis computer program, the automatic parsing procedure comprising automatically searching for parameter values of parameters of at least one predetermined list of parameters of interest in the program cycles of the synthesis computer program; and
ii. (denoted by reference number 154) automatically assembling a matrix of parameters comprising, for the program cycles of the synthesis computer program, the parameters of interest and the corresponding parameter values of the parameters of interest.

The method may further comprise the following optional steps:
iii. (denoted by reference number 156) automatically displaying the matrix of parameters on a display;
iv. (denoted by reference number 158) applying an automatic error detection step to the matrix of parameters, the automatic error detection step comprising subjecting the matrix of parameters to at least one automatic plausibility check; and
v. (denoted by reference number 160) an automatic error correction step, the automatic error correction step comprising automatically correcting at least one error in the program code detected in the automatic error detection step.

Examples of the program code and/or the matrix of parameters, specifically obtained from a program code with and without errors, are provided below.

FIG. 3 shows a flow chart of an exemplary embodiment of a method of synthesizing at least one oligonucleotide. The method comprises the following steps which, as an example, may be performed in the given order. It shall be noted, however, that a different order is also possible. Further, it is also possible to perform one, more than one or even all of the method steps once or repeatedly. Further, it is possible to perform two or more of the method steps simultaneously or in a timely overlapping fashion. The method may comprise further method steps which are not listed.

The method comprises:
a. (denoted by reference number 162) providing a program code of a synthesis computer program, the synthesis computer program being configured for computer-controlling at least one automatic synthesizer, the synthesis computer program having a plurality of program cycles for computer-controlling the automatic synthesizer to sequentially synthesize the oligonucleotide by using a sequence of synthesis cycles;
b. (denoted by reference number 150) applying the method of verifying the program code according to the present invention, such as according to the exemplary embodiment shown in FIG. 2 and/or according to any other embodiment disclosed herein; and
c. (denoted by reference number 164) computer-controlling the at least one an automatic synthesizer 114 to automatically synthesize the at least one oligonucleotide.

The method may further comprise:
d. (denoted by reference number 166) at least one correction step, comprising amending at least one parameter value of at least one parameter of interest in accordance with at least one result of step b.

Further, in step c., the at least one automatic synthesizer 114 as shown in FIG. 1 may be used. Thus, step c. may comprise computer-controlling at least the switchable valves 124 and the pumps 118. Specifically, step c. may comprise computer-controlling at least one of the further elements, such as the amidite valves 126, 128, the reagent valve 130, the column inlet valve 132, the column outlet valve 134, the recycle valve 136 and/or the waste valve 140.

### EXAMPLES

In the following, exemplary embodiments of a program code (Example 1) to be used in the method of verifying a program code of a synthesis computer program, matrices of parameters (Examples 2 and 3) obtained by performing the method of verifying a program code of a synthesis computer program are given.

### Example 1: Exemplary Program Code

Example 1 refers to an exemplary program code to be used in the method of verifying a program code of a synthesis computer program. The program code is a Unicorn method file, specifically a binary file comprising method procedure itself, instrument parameters, metadata, etc. The program code comprises a digital header listing names of the blocks of program code with corresponding byte addresses. The sequence of instructions appears in section “METHOD” and can be read out as text.

The program code comprises a main sequence of instructions, i.e. a main procedure:

MAIN_SEPARATION
0.00 Base CV, #Column_Volume {ml}, Any
0.00 Block Normal, START_parameters
0.00 Block Normal, Column_wash
0.00 Block Normal, Cycle01_mGo_only_detrit
0.00 Block Normal, Cycle02_mGs
0.00 Block Normal, Cycle03_mCs
[....]
0.00 Block Normal, Cycle21_fUs
0.00 Block Normal, Cycle22_pUs_no_cap_detrit
0.00 Block Normal, Backbone_deprotection
0.00 Block Normal, Method_end
0.00 End_Method
END_SEPARATION

Further, the program code comprises definitions of blocks of program code refer to in the main procedure:

SUB_SEPARATION START_parameters
0.00 Base Time
0.00 Block Normal, Column_Number
0.00 Block Normal, UV_Detrit
0.00 Block Normal, Purge_Detritylation_Autozero
0.00 Scale #Weight_of_Support {g}, #Loading_of_Support {umol/g}
0.00 Coldiameter #Column_Diameter {mm}
0.00 End_block
END_SEPARATION

SUB_SEPARATION Column_Number
0.00 Base Time
0.00 Column #Column_Number
0.10 End_Block
END_SEPARATION

SUB_SEPARATION UV_Detrit
0.00 Base Time
0.00 UV_Wavelength #UV_Detritylation {nm}, 280 {nm}, OFF {nm}
0.10 End_block
END_SEPARATION

Each block of program code referred to in the main sequence or from inside other blocks of program code is declared by “SUB_SEPARATION keyword” and end with “END_SEPARATION”. For example, calling the block of program code for cycle 03 in the main procedure comprise the instructions “0.0 Block Normal, Cycle03_mCs” and refers to the following block of program code comprised by the program code:

SUB_SEPARATION Cycle03_mCs
0.00 Base Time
0.00 Set_Mark “# Cycle03 mCs #”
0.05 Block Normal, Coupling_v1_C_10min
0.05 Block Normal, Thiolation
0.05 Block Normal, Capping
0.05 Block Normal, Detritylation_Amount_6CV
END_SEPARATION

The subprocedures or the blocks of program code may refer to further blocks of program code being defined in the program code. Each line in the block (except for first and last) starts with the position mark, followed by command and parameters. The position mark may be in time units, e.g. minutes, volume, e.g. milliliters or CV, e.g. column volumes. The units are defined by mandatory first line in the block by instructions “Base Time”, “Base Volume”, “Base CV” or “Base SameAsMain”, wherein the latter defines the same units as in main procedure.

The blocks of program code are defined by name and are entered in the program code once. References to blocks of program code from multiple places in the program code refer to the same entry. That means that the program code is not sequential and that changing parameters in a block of program code in one place will change them also in other places where this block is called.

### Example 2: Matrix of Hypothetical Parameters Obtained from a Program Code without Errors

Example 2 shows an exemplary embodiment of a matrix of hypothetical parameters obtained by performing the method of verifying a program code of a synthesis computer program, for example being performed with the program code of Example 1. The matrix of parameters in this example is obtained from a program code free of errors. The rows of the matrix denote the program cycles, whereas the columns of the matrix denote the parameters of interest and the corresponding parameter values of the parameters of interest. Other types of visualization are generally also feasible.

TABLE 1
Exemplary matrix of hypothetical parameter values obtained from a program code free of errors

| Unit | Unit | | | | SetpointA | SetpointB | FlowA | FlowB |
|---|---|---|---|---|---|---|---|---|
| Op Number | Operation | Cycle | ReagentA | ReagentB | [CV] | [CV] | [mL/min] | [mL/min] |
| U02 | Column | 0 | Amidite Z | Extra_4.6 | 2.0 | 2.0 | 32.2 | 32.2 |
| | wash | | | | | | | |
| U03 | Detritylation | 1-2 | Detrit_3.7 | Extra_4.6 | 1.5 | 3 | 16.6 | 38.7 |
| U03 | Detritylation | 3-4, 6-10, 17 | Detrit_3.7 | Extra 4.6 | 1.25 | 3.2 | 16.6 | 38.7 |
| U03 | Detritylation | 5, 11-15 | Detrit_3.7 | Extra_4.6 | 1.2 | 2.5 | 16.6 | 38.7 |
| U03 | Detritylation | 16, 18-19 | Detrit_3.7 | Extra_4.6 | 2.8 | 1.2 | 16.6 | 38.7 |
| U03 | Detritylation | 20 | Detrit_3.7 | Extra_4.6 | 1.46 | 2.5 | 16.6 | 38.7 |
| U03 | Detritylation | 21 | Detrit_3.7 | Extra 4.6 | 3.4 | 1.2 | 16.6 | 38.7 |
| U04 | Detritylation | 1-21 | ACN_Amidites_3.1 | ACN_Reag_4.1 | 2.0 | 2.0 | 27.6 | 27.6 |
| | Wash | | | | | | | |
| U05 | Coupling | 2-3, 5, 11-13, | Amidite | Activator | 0.5 | 0.25 | 6.5 | 6.5 |
| | Load | 15, 17, 20 | | | | | | |
| U05 | Coupling | 2-3, 5, 11-13, | Amidite ACN | Activator | 0.05 | 0.03 | 6.5 | 6.5 |
| | Push | 15, 17, 20 | | | | | | |
| U05 | Coupling | 2-21 | — | — | 0.0 | 0.8 | 0 | 13.5 |
| | Recycle | | | | | | | |
| U05 | Coupling | 2-21 | — | — | 0.0 | 2.7 | 0 | 60.6 |
| | Recycle | | | | | | | |
| U05 | Coupling | 4, 6-10, 14, | Amidite | Activator | 0.5 | 0.2 | 6.5 | 7.2 |
| | Load | 16, 18-19, 21 | | | | | | |
| U05 | Coupling | 4, 6-10, 14, | Amidite ACN | Activator | 0.04 | 0.01 | 6.5 | 7.2 |
| | Push | 16, 18-19, 21 | | | | | | |
| U05 | Coupling | 22 | Amidite | Activator | 0.5 | 0.8 | 3.9 | 9.2 |
| | Load | | | | | | | |
| U05 | Coupling | 22 | Amidite ACN | Activator | 0.5 | 0.1 | 3.9 | 9.2 |
| | Push | | | | | | | |
| U05 | Coupling | 22 | — | — | 0.0 | 10 | 0 | 60.6 |
| | Recycle | | | | | | | |
| U06 | Coupling | 2-22 | ACN_Amidites_3.1 | ACN_Reag_4.1 | 0.8 | 0.8 | 32.2 | 32.2 |
| | Wash | | | | | | | |
| U07 | Thiolation | 2-3, 20-22 | ACN_Amidites_3.1 | Thiolation | 0.0 | 0.5 | 0 | 34.9 |
| U07 | Thiolation | 2-3, 20-22 | — | — | 0.0 | 5 | 0 | 67.3 |
| U08 | Thiolation | 2-3, 20-22 | ACN_Amidites_3.1 | ACN_Reag_4.1 | 2 | 2 | 33.7 | 33.7 |
| | Wash | | | | | | | |
| U09 | Capping | 2-21 | Cap_A | Cap_B | 2 | 2 | 26.9 | 26.9 |
| U09 | Capping | 2-21 | ACN_Amidites_3.1 | ACN_Reag_4.1 | 0.3 | 0.8 | 26.9 | 26.9 |
| U10 | Capping | 2-21 | Amidite Z | Extra_4.6 | 1 | 1 | 32.2 | 32.2 |
| | Wash | | | | | | | |
| U11 | Oxidation | 4-19 | ACN_Amidites_3.1 | Ox | 0.0 | 5 | 0 | 34.9 |
| U11 | Oxidation | 4-19 | ACN_Amidites_3.1 | ACN_Reag_4.1 | 0.0 | 1.0 | 0 | 34.9 |
| U12 | Oxidation | 4-19 | ACN_Amidites_3.1 | ACN_Reag_4.1 | 1.0 | 1.0 | 33.7 | 33.7 |
| | Wash | | | | | | | |
| U13 | Backbone | 23 | ACN_Amidites_3.1 | DEA | 0.0 | 3.8 | 0 | 8.4 |
| | deprotection | | | | | | | |
| U13 | Backbone | 23 | — | — | 0.0 | 11.3 | 0 | 8.4 |
| | deprotection | | | | | | | |
| U13 | Backbone | 23 | ACN_Amidites_3.1 | ACN_Reag_4.1 | 4.0 | 4.0 | 33.7 | 33.7 |
| | deprotection | | | | | | | |

### Example 3: Matrix of Hypothetical Parameters Obtained from a Program Code with Errors

Example 3 shows an exemplary embodiment of a matrix of hypothetical parameters obtained by performing the method of verifying a program code of a synthesis computer program, for example being performed with the program code of Example 1. The matrix of parameters in this example is obtained from a program code comprising one or more errors. Errors are highlighted in bold letters in Table 2.

TABLE 2
Exemplary matrix of hypothetical parameter values obtained from a program code with errors

| Unit | Unit | | | | SetpointA | SetpointB | FlowA | FlowB |
|---|---|---|---|---|---|---|---|---|
| Op Number | Operation | Cycle | ReagentA | ReagentB | [CV] | [CV] | [mL/min] | [mL/min] |
| U02 | Column | 0 | Amidite Z | Extra_4.6 | 2 | 2 | 32.2 | 32.2 |
| | wash | | | | | | | |
| U03 | Detritylation | 1-2 | Detrit_3.7 | Extra_4.6 | 1.5 | 3 | 16.6 | 38.7 |
| U03 | Detritylation | 3-4, 6-10, 17 | Detrit_3.7 | Extra_4.6 | 1.25 | 3.2 | 16.6 | 38.7 |
| U03 | Detritylation | 5, 11-15 | Detrit_3.7 | Extra_4.6 | 1.2 | 2.5 | 16.6 | 38.7 |
| U03 | Detritylation | 16, 18-19 | Detrit_3.7 | Extra_4.6 | 2.8 | 1.2 | 16.6 | 38.7 |
| U03 | Detritylation | 20 | Detrit_3.7 | Extra_4.6 | 1.46 | 2.5 | 16.6 | 38.7 |
| U03 | Detritylation | 21 | Detrit_3.7 | Extra_4.6 | 3.4 | 1.2 | 16.6 | 38.7 |
| U04 | Detritylation | 1-21 | ACN_Amidites_3.1 | ACN_Reag_4.1 | 2 | 2 | 27.6 | 27.6 |
| | Wash | | | | | | | |
| U05 | Coupling | 2, 5, 11-13, | Amidite | Activator | 0.5 | 0.25 | 6.5 | 6.5 |
| | Load | 15, 17, 20 | | | | | | |
| U05 | Coupling | 2, 5, 11-13, | Amidite ACN | Activator | 0.05 | 0.03 | 6.5 | 6.5 |
| | Push | 15, 17, 20 | | | | | | |
| U05 | Coupling | 2-21 | Amidite ACN | Activator | 0 | 0.8 | 0 | 13.5 |
| | Recycle | | | | | | | |
| U05 | Coupling | 2-21 | Amidite ACN | Activator | 0 | 2.7 | 0 | 60.6 |
| | Recycle | | | | | | | |
| U05 | Coupling | 3 | Amidite | Activator | 0.005 | 0.0025 | 6.5 | 6.5 |
| | Recycle | | | | | | | |
| U05 | Coupling | 3 | Amidite ACN | Activator | 0.025 | 0.025 | 6.5 | 6.5 |
| | Recycle | | | | | | | |
| U05 | Coupling | 4, 6-10, 14, | Amidite | Activator | 0.5 | 0.2 | 6.5 | 7.2 |
| | Load | 16, 18-19, 21 | | | | | | |
| U05 | Coupling | 4, 6-10, 14, | Amidite ACN | Activator | 0.04 | 0.01 | 6.5 | 7.2 |
| | Recycle | 16, 18-19, 21 | | | | | | |
| U05 | Coupling | 22 | Amidite | Activator | 0.5 | 0.8 | 3.9 | 9.2 |
| | Load | | | | | | | |
| U05 | Coupling | 22 | Amidite ACN | Activator | 0 . . . 5 | 0.1 | 3.9 | 9.2 |
| | Push | | | | | | | |
| U05 | Coupling | 22 | — | — | 0 | 10 | 0 | 60.6 |
| | Recycle | | | | | | | |
| U06 | Coupling | 2-22 | ACN_Amidites_3.1 | ACN_Reag_4.1 | 0.8 | 0.8 | 32.2 | 32.2 |
| | Wash | | | | | | | |
| U07 | Thiolation | 2-3, 20-22 | ACN_{—}Amidites_{—}3.1 | ACN_{—}4.4 | 0 | 0.5 | 0 | 34.9 |
| U07 | Thiolation | 2-3, 20-22 | — | — | 0 | 5 | 0 | 67.3 |
| U08 | Thiolation | 2-3, 20-22 | ACN_Amidites_3.1 | ACN_Reag_4.1 | 2 | 2 | 33.7 | 33.7 |
| | Wash | | | | | | | |
| U09 | Capping | 2-21 | Cap_A | Cap_B | 2 | 2 | 26.9 | 26.9 |
| U09 | Capping | 2-21 | ACN_Amidites_3.1 | ACN_Reag_4.1 | 0.3 | 0.8 | 26.9 | 26.9 |
| U10 | Capping | 2-21 | Amidite Z | Extra_4.6 | 1 | 1 | 32.2 | 32.2 |
| | Wash | | | | | | | |
| U11 | Oxidation | 4-19 | ACN_Amidites_3.1 | Ox | 0 | 5 | 0 | 34.9 |
| U11 | Oxidation | 4-19 | ACN_Amidites_3.1 | ACN_Reag_4.1 | 0 | 1 | 0 | 34.9 |
| U12 | Oxidation | 4-19 | ACN_Amidites_3.1 | ACN_Reag_4.1 | 1 | 1 | 33.7 | 33.7 |
| | Wash | | | | | | | |
| U13 | Backbone | 23 | ACN_Amidites_3.1 | DEA | 0 | 3.75 | 0 | 8.4 |
| | deprotection | | | | | | | |
| U13 | Backbone | 23 | — | — | 0 | 11.25 | 0 | 8.4 |
| | deprotection | | | | | | | |
| U13 | Backbone | 23 | ACN_Amidites_3.1 | ACN_Reag_4.1 | 4 | 4 | 33.7 | 33.7 |
| | deprotection | | | | | | | |

In this example, errors in the program code can be detected by performing the automatic plausibility check on the matrix of parameters. For example, Table 3 shows exemplary errors in the program code, their effect on the synthesis of the oligonucleotide and how these errors can be detected in the matrix of parameters of Table 2:

TABLE 3
Exemplary aspects of the automatic plausibility check

| Error type | Effect on the synthesis | Detected by |
|---|---|---|
| Wrong units (e.g., “Base volume” | Wrong amounts of reagents used. | An extra line appears in the matrix of |
| instead of “Base time”) | Sometimes may stop or stall the | parameters with erroneous values. |
| | synthesis | (see Table 2, where a separate line for |
| | | Coupling for Cycle 3 appears with |
| | | Setpoint values lower than in normal |
| | | method). |
| Setting of reactant or solvent is | Synthesis failure | Wrong entries in “Reactant” columns |
| omitted | | in the matrix of parameters. This can |
| | | be easily detected when compared to |
| | | other matrices of parameters: |
| | | In Table 2, U07 Thiolation, Reactant |
| | | B is ACN_4.4 instead of “Thiolation”. |
| Wrong setting of a valve (usually, the | Too much reagent and solvent is used - | Erroneous values in the matrix of |
| valve to waste or recycle) | may result in stalling the synthesis | parameters: Detectable by comparison |
| | | with other matrices of parameters. |
| | | Coupling Recycle normally runs in a |
| | | loop and consumes no reagents (“—” in |
| | | reagent columns). In both recycling |
| | | steps in U05 Coupling Recycle, the |
| | | matrix of parameters shows reagents, |
| | | as the valve was not switched (see |
| | | Table 2). |
| Flow not set to zero at the end of | Sometimes may result in high | The affected operation has higher |
| operation | pressure in one of the pumps, causing | parameter values and wrong |
| | emergency halt | regents/solvents. The operation next is |
| | | missing in the matrix of parameters. |
| | | In Table 2, the flow was not stopped |
| | | at the end of Coupling Push in Cycle |
| | | 3. Coupling Push parameters for |
| | | Cycle 3 appear in separate line, |
| | | erroneously labeled “Coupling |
| | | Recycle”. |

Thus, in this example, the automatic plausibility check as shown in Table 3 may detect one or more of the following errors of the matrix of parameters of Table 2: an extra line appearing in the matrix of parameters with erroneous values; wrong entries in “Reactant” columns in the matrix of parameters; erroneous values in the matrix of parameters; a next operation next is missing in the matrix of parameters.

### Example 4: Comparison of Input Parameters and Highlighting

In practical use, synthesis computer programs (synthesis programs) of synthesis devices may be programmed de novo; however, frequently, new synthesis programs are generated by modifying existing synthesis programs. Thus, a comparison e.g. of the previous and the new synthesis program facilitates verification that the parameters intended to change, and only the parameters intended to change, were modified.

Thus, instead of or in addition to error highlighting as detailed in Example 3, also selection of a comparison method was enabled, e.g. of a method used previously or a reference method and a list of method steps of the method of interest was output, in which differences to the comparison method were highlighted, thus improving error detection.

### Example 5: Reagent Calculation

In the methods described herein, also the reagent volume of a given step, as well as the number of cycles in which the reagent is used, can be parameters of interest. From these parameters, automatically the volume of reagent required for performing the method was calculated. It will be understood that this calculation may be performed for one or more educts, auxiliary and washing reagents. Thus, in an embodiment, required volumes of all reagents used in the method were calculated, preventing interruptions of the methods caused by lack of reagent.

### Example 6: Verifying a Set of Methods

As the skilled person will understand in view of the description herein, e.g. in method optimization, a variety of parameters may have to be optimized, of which at least a fraction may be mutually interdependent; thus, it may be necessary to perform optimization with a large number of methods having combinatorial sets of parameters. In such case, it is particularly difficult to ensure that all combinations were programmed as originally planned, e.g. in a matrix of design of experiments (DOE) values.

To that end, in an embodiment a structured array of parameter scores was used. On an exemplary basis, in case a set of three parameters A, B, and C, were optimized, a strucured array comprised the score of parameter A in first position, the score of parameter B in second position, and the score of parameter C in third position. Also on an exemplary basis, parameters A and B were used as a low values represented by “−”, as medium values represented by “o”, and high values represented by “+”. As will be understood by the skilled person, the actual values or ranges represented by the aforesaid symbols may be different for parameters A and B. Also on an exemplary basis, parameter C was used as four values, represented by “1”, “2”, “3”, and “4”, respectively, Thus a method using a low value of parameter A, a medium value of parameter B, and the highest value of parameter C, would be represented by a string “-o4”. In conclusion, a target pattern for a set of methods using all combinations of a low value of parameter A, a medium value of parameter B, and all values of parameter C in increasing order could be represented as the following target pattern:
o1
o2
o3
o4

In the verification method, parameters A, B, and C were parameters of interest and a verification matrix of parameters was generated for the programmed set of synthesis methods. Verification comprised comparing the verification matrix of parameters to the aforesaid target pattern. As will be appreciated, detection of deviations in such patterns is easier and more reliable than comparing e.g. parameter lists or even lists of method steps.

### LIST OF REFERENCE NUMBERS

110 system
112 processor
114 automatic synthesizer
116 synthesis column
118 pump
120 connectors
122 chemical supply
124 switchable valve
126 amidite valve
128 amidite valve
130 reagent valve
132 column inlet valve
134 column outlet valve
136 recycle valve
138 pH-C-meter
140 waste valve
142 waste system
144 UV-absorption sensor
146 flow restrictor
148 column bypass
150 method of verifying a program code of a synthesis computer program
152 applying an automatic parsing procedure
154 automatically assembling a matrix of parameters
156 automatically displaying the matrix of parameters
158 applying an automatic error detection step
160 automatic error correction step
162 providing a program code of a synthesis computer program
164 computer-controlling the automatic synthesizer
166 correction step

1. A method of verifying a program code of a synthesis computer program, the synthesis computer program being configured for computer-controlling an automatic synthesizer to automatically synthesize at least one oligonucleotide, the synthesis computer program having a plurality of program cycles for computer-controlling the automatic synthesizer to sequentially synthesize the oligonucleotide by using at least one sequence of synthesis cycles, the method comprising:
i. applying an automatic parsing procedure to the program code of the synthesis computer program, the automatic parsing procedure comprising automatically searching for parameter values of parameters of at least one predetermined list of parameters of interest in the program cycles of the synthesis computer program; and
ii. automatically assembling a matrix of parameters comprising, for the program cycles of the synthesis computer program, the parameters of interest and the corresponding parameter values of the parameters of interest.

2. The method according to claim 1, wherein said synthesis cycles comprise at least two elongation cycles wherein a nucleotide residue is added to the oligonucleotide chain, wherein the parameters of interest are identical for at least a fraction of the program cycles controlling the elongation cycles, such that the matrix comprises the parameter values for identical parameters of different program cycles of the program code.

3. The method according to claim 1, the method further comprising:
automatically displaying the matrix of parameters on a display.

4. The method according to claim 1, wherein the predetermined list of parameters of interest comprises at least one parameter of interest selected from the group consisting of: a type of a chemical; a volume of a chemical; a flow rate of a chemical; a temperature of a chemical; a pump pressure of at least one pump; and a length of a particular synthesis step.

5. The method according to claim 1, the method further comprising:
applying an automatic error detection step to the matrix of parameters, the automatic error detection step comprising subjecting the matrix of parameters to at least one automatic plausibility check, wherein said automatic plausibility check comprises at least one of the following:
comparing a pattern of the matrix of parameters with at least one predetermined target pattern;
detecting missing parameter values in the matrix of parameters;
detecting incorrect units;
detecting parameter values out of a predetermined range;
detecting missing lines in the matrix;
detecting additional lines in the matrix;
identifying parameters of interest of the program cycles being listed in dif-ferent lines of the matrix which should be listed in a combined line of the matrix;
detecting the selection of correct reagents;
detecting the selection of correct solvents; or
detecting the selection of correct starting materials.

6. The method according to claim 5, further comprising an automatic output of warning information to a user if the automatic error detection step detects an error in the program code.

7. The method according to claim 5, further comprising an automatic prevention of the synthesis computer program from being executed on said automatic synthesizer if the automatic error detection step detects an error in the program code.

8. The method according to claim 5, wherein, in the automatic error detection step, at least one of the following errors is detected: incorrect units of the parameters; an omitted setting of at least one chemical; an incorrect set-ting of at least one valve; or a flow of at least one chemical not being set to zero at the end of the program code.

9. The method according to claim 5, further comprising:
an automatic error correction step, the automatic error correction step comprising automatically correcting at least one error in the program code detected in the automatic error detection step.

10. The method according to claim 1, wherein the program code comprises a header and a text section following the header, and further comprising an automatic detection of the text section in the program code, wherein the automatic parsing procedure is performed on the text section.

11. The method according to claim 1, wherein the matrix of parameters comprises a comparison of said matrix of parameters to a reference matrix of parameters.

12. The method according to claim 1, wherein automatically displaying the matrix of parameters on the display comprises displaying on the display a representation of method steps comprising a comparison of method steps to a reference list of method steps.

13. The method according to claim 1, further comprising:
automatically calculating a total required amount of at least one reagent based on said parameter values.

14. A method of verifying a set of at least two program codes of synthesis computer programs, each synthesis computer program being configured for computer-controlling an automatic synthesizer to automatically synthesize at least one oligonucleotide, each synthesis computer program having a plurality of program cycles for computer-controlling the automatic synthesizer to sequentially synthesize the oligonucleotide by using at least one sequence of synthesis cycles, the method comprising:
(I) applying an automatic parsing procedure to the program code of each of said synthesis computer programs, the automatic parsing procedure comprising automatically searching for parameter values of parameters of at least one predetermined list of parameters of interest in the program cycles of the synthesis computer programs;
(II) automatically assembling a verification matrix of parameters comprising, for the program cycles of each of the synthesis computer programs, (i) the parameters of interest and the corresponding parameter scores of the parameters of interest, or (ii) a structured array of parameter scores of said parameters of interest; and
(III) comparing a pattern of the verification matrix of parameters with at least one predetermined target pattern.

15. (canceled)

16. (canceled)

17. (canceled)

18. A method of synthesizing at least one oligonucleotide, the method comprising:
providing the program code of the synthesis computer program;
applying the method of claim 1; and
computer-controlling at least one automatic synthesizer to automatically synthesize the at least one oligonucleotide.

19. The method according to claim 18, the method further comprising performing at least one correction step before computer-controlling the at least one automatic synthesizer to automatically synthesize the at least one oligonucleotide, the at least one correction step comprising amending at least one parameter value of at least one parameter of interest in accordance with at least one result of applying the method of claim 1.

20. The method of synthesizing according to claim 18, wherein the at least one oligonucleotide comprises at least one oligonucleotide selected from the group consisting of: a DNA oligonucleotide, an RNA oligonucleotide, and an LNA oligonucleotide.

21. The method according to claim 11, wherein automatically assembling the matrix of parameters further comprises highlighting differences between the matrix of parameters and the reference matrix of parameters.

22. At least one non-transitory computer-readable storage medium comprising a plurality of instructions stored thereon that, in response to execution by a computing system, causes the computing system to:
apply an automatic parsing procedure to a program code of a synthesis computer program, the synthesis computer program being configured for computer-controlling an automatic synthesizer to automatically synthesize at least one oligonucleotide, the synthesis computer program having a plurality of program cycles for computer-controlling the automatic synthesizer to sequentially synthesize the oligonucleotide by using at least one sequence of synthesis cycles, wherein the automatic parsing procedure comprises to automatically search for parameter values of parameters of at least one predetermined list of parameters of interest in the program cycles of the synthesis computer program; and
automatically assemble a matrix of parameters comprising, for the program cycles of the synthesis computer program, the parameters of interest and the corresponding parameter values of the parameters of interest.
